(19) United States
(12) Patent Application Publication
(10) Pub. No.: US 2021/0003563 A1
(43) Pub. Date: Jan. 7, 2021

(54) PATIENT-SPECIFIC CANCER THERAPY SCREENING AND METHODS OF TREATMENT

(71) Applicant: Avera McKennan, Sioux Falls, SD (US)

(72) Inventors: Nandini DEY, Sioux Falls, SD (US); Pradip DE, Sioux Falls, SD (US)

(73) Assignee: Avera McKennan, Sioux Falls, SD (US)

(21) Appl. No.: 16/875,910

(22) Filed: May 15, 2020

Related U.S. Application Data
(60) Provisional application No. 62/848,461, filed on May 15, 2019.

(51) Int. Cl. G01N 33/50; C12N 5/078; A61K 31/337

(52) U.S. Cl. CPC G01N 33/5047; C12N 5/0634; C12N 2500/02; C12N 2502/30; A61K 31/337

(57) ABSTRACT

Methods, systems, and devices for screening chemotherapeutic drug(s) on patient specific tissue and the related methods of treating cancer are provided. Candidate chemotherapeutic drug(s) may be tested on samples of resected tumor and/or tumor-adjacent normal tissue from the patient ex-vivo. In some cases, the tumor tissue and tumor-adjacent normal tissue samples are separately co-cultured ex-vivo in the presence of immune cells isolated from the same patient. The tumor and tumor-adjacent normal tissue is evaluated post-treatment for markers of efficacy of the candidate regimens. A chemotherapeutic regimen(s) that provide optimal treatment of the patient's specific cancer may then be considered by the oncologist to be administered to the patient.

### 1. BACKGROUND

Cancer is a genetic disease. These genetic alteration(s) are responsible for the oncogenesis, treatment response, clinical outcome, resistance to therapy, and progression of the disease. Broadly, the treatment of cancer by drugs may be classified into three categories, chemotherapy, targeted therapy, and immune-therapy. Chemotherapy is one of the most common methods of treating cancer and is widely employed in the battle against a variety of cancers in patients all over the world. Different chemotherapeutic agents may be designed to treat cancerous tumors at a specific organ site. However, almost all chemotherapeutic agents have collateral toxic effect on normal dividing cells of the patients' body. A second category of therapy, targeted therapy, may been employed where the drug targets the specific protein product(s) and/or cell signal pathway(s) of the altered genes in patients. As a result, it is desirable to employ cancer treatment methods selectively to maximize effects on the patient's cancer while minimizing the impacts of the treatment on the patient.

Several types of models had been employed by researchers for several decades to test the efficacy of chemotherapeutic, targeted and immune-therapy drugs, alone or in combination. The success of a test (to evaluate the efficacy of a drug combination) depends on the accurateness of the model used for the test.

### 2. SUMMARY

The evolution of omics in cancer research opened the flood gate of information in recent years. Omics may refer to a field of study in biology ending in -omics, such as genomics, proteomics or metabolomics. Omics may also refer to the collective technologies used to explore the roles, relationships, and actions of the various types of molecules that make up the cells of an organism. Studies concluded that cancer is not only a genetic disease but also all tumors of a particular organ-site(s) are genetically different from patient to patient. The advent of this concept introduced the concept of personalized/tailored medicine in the world of cancer therapy. Thus personalized medicine demands personalized model to test cancer therapy drugs to either test the drug efficacy or to identify markers (e.g., genetic markers) in a patient.

Methods, systems, and devices are described for treating cancer on a patient-specific basis in a time-efficient manner. Patients are treated with a chemotherapeutic regimen identified by testing candidate regimens on samples of tumor tissue obtained from a patient by biopsy or resection. In some examples of the methods of the invention, the samples may include tumor tissue as well as metastatic tumor tissue (if received from the patient on a case to case basis) and tumor-adjacent normal tissue from the patient that may then be cultured ex-vivo. In some cases, the ex-vivo culture may also include immune cells such as leukocytes, e.g., CD3+ T-cells and/or CD14−/CD15+/CD16+ neutrophils (“N-cells”) isolated from the same patient. These immune cells may be obtained and characterized from a blood sample from the patient. That blood sample may be taken at or close to the time that the tumor tissue is obtained.

In some examples, one culture may include tumor tissue, another culture may include tumor-adjacent normal tissue, another culture may include metastatic tumor tissue, and optionally another co-culture may include tumor tissue and immune cells. The chemotherapeutic regimen may be administered under a range of oxygenation conditions, including normoxia, but also under hypoxia, re-oxygenation and hyperoxia. In some instances, such oxygenation conditions may more closely resemble the conditions inside the source tumor tissue.

The tissue is evaluated post-treatment of a chemotherapeutic regimen for markers of efficacy of the candidate regimens. A regimen or regimens which provide optimal treatment of the patient's specific cancer may then be administered to the patient based on the clinical decision of the oncologist based on this personalized test of the patient's tumor tissue, normal tissue, and immune cells. Thus, a robust precision cancer care platform of the patient-centric functional model by using patient's resected tissue samples in the ex-vivo setting for culture and immune co-culture purpose has been established. This co-culture model achieves an ideal and unique model system that is personalized for an individual patient, their tumor tissue, their tumor-adjacent normal tissue, and their peripheral immune cells to participate in drug testing outside patient's body.

In addition to the above, several additional steps may be included to provide additional information for medical personnel to use in evaluating treatment protocols. These include any one or more of the following optional steps:

The patient's blood sample may further be tested for the presence of circulating tumor cells (“CTCs”), to provide medical personnel with another data point relative to longer-term aspects of a suitable treatment regimen. CTCs may be tested for using techniques known in the art, by CellSieve™ CTC Enumeration kit-Creatv Microtech. CTCs were isolated on CellSieve™ Precision Microfilters [Creatv MicroTech, Inc., Potomac, Md.].

The patient's blood sample may further be tested for the presence of circulating cancer associated macrophage like cells (“CAMLs”) to provide medical personnel with another data point relative to longer-term aspects of a suitable treatment regimen. CAMLs have been isolated from blood samples using technologies by CellSieve™ CTC Enumeration kit-Creatv Microtech. CTCs were isolated on CellSieve™ Precision Microfilters [Creatv MicroTech, Inc., Potomac, Md.]. In some instances, both CTCs and CAMLs may be identified from the same blood sample from the patient.

In another optional set of steps, at least a portion of the tumor tissue sample taken from the patient may further be subjected to genomic testing to identify known gene alterations associated with specific cancer types. This process typically requires more time for completion than the co-culturing steps discussed herein. As a result, separately, and in some instances, in parallel to the co-culture steps, a portion of the tumor tissue sample is cultured to prepare patient-derived cells to preserve them to allow testing of the sample against the treatment(s) recommended by both the co-culture steps taught herein and/or the treatment recommended as a result of the genomic testing. In some instances, the portion of the tumor tissue sample is cultured to prepare patient-derived cells. The culture process described herein prepares a culture of cancer-associated fibroblasts (from tumor tissue) and normal fibroblasts (from normal tissue) which can be maintained as needed to allow testing of genetic testing-recommended and/or co-culture-recommended therapies. This culture process reduces the tissue sample to just fibroblasts—normal and cancer associated fibroblasts, each of which can be cultured through multiple passages to selectively preserve the cancer-associated fibroblasts for future drug-testing.

Genomic analysis of a tumor may be a key approach for drug matching in the era of precision medicine, but translational scientists may benefit from a patient-centric functional model that is time sensitive, feasible in clinics, and cost-effective to test certain combination(s) of drugs. In recent years, immunotherapies have transformed the landscape of cancer therapy in a selected population of patients and certain cancer types. In recent years commercial companies have ventured out to use humanized mouse models to test immune-therapy drugs which are labor intensive, cost intensive and limited to only certain types of cancers. These models are humanized but not personalized and take months of time with an element of uncertainty. There is no patient-centric functional in vitro or in vivo model system that is time sensitive, feasible in clinics, and cost-effective to test immune checkpoint inhibitor(s), and there is no in vitro or in vivo model system to test immune checkpoint inhibitor(s) with tumor-specific targeted therapies. Thus, predictive preclinical models are needed to drive the expansion of rational immunotherapy drug development and minimize failures in clinical trials.

The ex-vivo culture and immune-co-culture platform described herein is a unique personalized functional model that integrates a tumor's microenvironment with patients' own immune cells in order to test the rationale combination of drugs outside patients' bodies. This personalized test may provide experimentally proven information within a short time frame (e.g., up to seven days) and in a cost-effective manner to oncologists that may be included in their clinical decision making of what drug therapy to use for the patient in a clinic.

A method of treating a patient with cancer is described. The method may include obtaining tumor- and tumor-adjacent normal tissues from a patient, exposing the tumor- and tumor-adjacent normal tissues to at least one cancer-therapeutic agent, culturing the tumor- and tumor-adjacent normal tissues with the at least one cancer-therapeutic agent, evaluating the tumor- and tumor-adjacent normal tissues for an effect of the at least one cancer-therapeutic agent, selecting at least one cancer-therapeutic agent based on at least one effect, and administering the at least one cancer-therapeutic agent to the patient.

An apparatus for treating a patient with cancer is described. The apparatus may include means for obtaining tumor- and tumor-adjacent normal tissues from a patient, exposing the tumor- and tumor-adjacent normal tissues to at least one cancer-therapeutic agent, culturing the tumor- and tumor-adjacent normal tissues with the at least one cancer-therapeutic agent, evaluating the tumor- and tumor-adjacent normal tissues for an effect of the at least one cancer-therapeutic agent, selecting at least one cancer-therapeutic agent based on at least one effect, and administering the at least one cancer-therapeutic agent to the patient.

In some examples of the method and apparatus described herein, the tumor- and/or tumor-adjacent normal tissues may be obtained from the patient by surgical resection, while in others, the tumor- and/or tumor-adjacent normal tissues are obtained from the patient by biopsy.

Some examples of the method and apparatus described herein may further include operations, features, means, or instructions for obtaining leukocytes from the patient, exposing the tumor- and tumor-adjacent normal tissues and the leukocytes to at least one cancer-therapeutic agent, and co-culturing the tumor- and tumor-adjacent normal tissues and the leukocytes with the at least one cancer-therapeutic agent. Leukocytes are involved in the body's response to foreign substance and/or disease, and leukocytes may be tested in an ex-vivo co-culture model with the at least one cancer-therapeutic agent, as described herein, to determine how a patient's immune system may respond to the at least one cancer-therapeutic agent.

In some examples of the method and apparatus described herein, the leukocytes may be obtained from peripheral blood of the patient from whom the tumor and tumor adjacent normal tissues are surgically resected. In some examples of the method and apparatus described herein, the leukocytes include CD3+ T-cells and/or CD14−/CD15+/CD16+ neutrophils from the patient. In some examples of the method and apparatus described herein, the leukocytes may be CD3+ T-cells and/or CD14−/CD15+/CD16+ neutrophils isolated from peripheral blood of the patient.

In some examples of the method and apparatus described herein, exposing the tumor- and tumor-adjacent normal tissues to at least one cancer-therapeutic agent may include operations, features, means, or instructions for culturing the tumor- and tumor-adjacent normal tissues with the at least one cancer-therapeutic agent for a period of at least about 6 hours.

In some examples of the method and apparatus described herein, exposing the tumor- and tumor-adjacent normal tissues to at least one cancer-therapeutic agent may include operations, features, means, or instructions for culturing the tumor- and tumor-adjacent normal tissues with the at least one cancer-therapeutic agent for a period of at least about 12 hours.

In some examples of the method and apparatus described herein, exposing the tumor- and tumor-adjacent normal tissues to at least one cancer-therapeutic agent may include operations, features, means, or instructions for culturing the tumor- and tumor-adjacent normal tissues with the at least one cancer-therapeutic agent for a period of at least about 24 hours.

In some examples of the method and apparatus described herein, exposing the tumor- and tumor-adjacent normal tissues to at least one cancer-therapeutic agent may include operations, features, means, or instructions for culturing the tumor- and tumor-adjacent normal tissues with the at least one cancer-therapeutic agent for a period of at least about 36 hours.

In some examples of the method and apparatus described herein, exposing the tumor- and tumor-adjacent normal tissues to at least one cancer-therapeutic agent may include operations, features, means, or instructions for culturing the tumor- and tumor-adjacent normal tissues with the at least one cancer-therapeutic agent for a period of at least about 48 hours.

In some examples of the method and apparatus described herein, exposing the tumor- and tumor-adjacent normal tissues to at least one cancer-therapeutic agent may include operations, features, means, or instructions for culturing the tumor- and tumor-adjacent normal tissues with the at least one cancer-therapeutic agent for a period of at least about 72 hours.

In some examples of the method and apparatus described herein, exposing the tumor- and tumor-adjacent normal tissues to at least one cancer-therapeutic agent may be done in a variety of oxygen environments. In some instances, this may better simulate or approximate the tumor environment. In some such, the tissues may be cultured in normoxia, i.e., a normal oxygen environment. In still others, the tissues may be cultured in hypoxic, re-oxygenation, or hyperoxic states.

Some examples of the method and apparatus described herein may further include operations, features, means, or instructions for evaluating the tumor- and tumor-adjacent normal tissues for an effect of the at least one cancer-therapeutic agent on the tumor- and tumor-adjacent normal tissues includes evaluating the tumor- and tumor-adjacent normal tissues for at least one of inhibition of proliferation, induction of apoptosis, inhibition of angiogenesis, effect on vascular mimicry, status of the pathway activation, and immune-status of the tumor- and tumor-adjacent normal tissues.

Some examples of the method and apparatus described herein may further include operations, features, means, or instructions for selecting the at least one cancer-therapeutic agent based on the at least one effect. This may include selecting at least one cancer therapeutic agent which induced apoptosis and/or inhibited proliferation of the tumor tissues while causing little or no damage to the tumor-adjacent normal tissue.

A method of patient-specific evaluation of cancer-therapeutic agents is described. The method may include obtaining tumor- and tumor-adjacent normal tissues from a patient, exposing the tumor- and tumor-adjacent normal tissues to at least one cancer-therapeutic agent, co-culturing the tumor- and tumor-adjacent normal tissues with the at least one cancer-therapeutic agent, evaluating the tumor- and tumor-adjacent normal tissues for an effect of the at least one cancer-therapeutic agent, and selecting at least one cancer-therapeutic agent based on at least one effect.

An apparatus for patient-specific evaluation of cancer-therapeutic agents is described. The apparatus may include means for obtaining tumor- and tumor-adjacent normal tissues from a patient, exposing the tumor- and tumor-adjacent normal tissues to at least one cancer-therapeutic agent, co-culturing the tumor- and tumor-adjacent normal tissues with the at least one cancer-therapeutic agent, evaluating the tumor- and tumor-adjacent normal tissues for an effect of the at least one cancer-therapeutic agent, and selecting at least one cancer-therapeutic agent based on at least one effect.

In some examples of the method and apparatus described herein, the tumor- and tumor-adjacent normal tissues may be obtained by surgical resection from the patient. In others, the tumor- and tumor-adjacent normal tissues may be obtained by biopsy from the patient. In some examples of the method and apparatus described herein, the tumor- and tumor-adjacent normal tissues may be resected or biopsied from the margin of a tumor of the patient.

Some examples of the method and apparatus described herein may further include operations, features, means, or instructions for obtaining leukocytes such as CD3+ T-cells and/or CD14−/CD15+/CD16+ neutrophils from the patient, exposing the tumor- and tumor-adjacent normal tissues and the CD3+ T-cells and/or CD14−/CD15+/CD16+ neutrophils to at least one cancer-therapeutic agent, and co-culturing the tumor- and tumor-adjacent normal tissues and the CD3+ T-cells and/or CD14−/CD15+/CD16+ neutrophils with the at least one cancer-therapeutic agent.

In some examples of the method and apparatus described herein, CD3+ T-cells and/or CD14−/CD15+/CD16+ neutrophils may be obtained from blood from the patient. In some examples of the method and apparatus described herein, CD3+ T-cells and/or CD14−/CD15+/CD16+ neutrophils may be obtained from a peripheral blood sample from the patient. In some examples of the method and apparatus described herein, CD3+ T-cells and/or CD14−/CD15+/CD16+ neutrophils may be obtained from blood from the patient by isolation using magnetic beads, and/or by other known characterization and/or separation methods.

In some examples of the method and apparatus described herein, the at least one cancer-therapeutic agent may be a chemotherapeutic agent, a pathway-targeted drug, an immune-modulatory drug, or any combination thereof.

In some examples of the method and apparatus described herein, the tumor- and tumor-adjacent normal tissues may be co-cultured with CD3+ T-cells and/or CD14−/CD15+/CD16+ neutrophils for a period of at least about 6 hours with the at least one cancer-therapeutic agent.

In some examples of the method and apparatus described herein, the tumor- and tumor-adjacent normal tissues may be co-cultured with CD3+ T-cells and/or CD14−/CD15+/CD16+ neutrophils for a period of at least about 12 hours with the at least one cancer-therapeutic agent.

In some examples of the method and apparatus described herein, the tumor- and tumor-adjacent normal tissues may be co-cultured with CD3+ T-cells and/or CD14−/CD15+/CD16+ neutrophils for a period of at least 24 hours with the at least one cancer-therapeutic agent.

In some examples of the method and apparatus described herein, the tumor- and tumor-adjacent normal tissues may be co-cultured with CD3+ T-cells and/or CD14−/CD15+/CD16+ neutrophils for a period of at least 48 hours with the at least one cancer-therapeutic agent.

In some examples of the method and apparatus described herein, the tumor- and tumor-adjacent normal tissues may be co-cultured with CD3+ T-cells and/or CD14−/CD15+/CD16+ neutrophils for a period of at least 72 hours with the at least one cancer-therapeutic agent.

Some examples of the method and apparatus described herein may further include operations, features, means, or instructions for evaluating the tumor- and tumor-adjacent normal tissues for an effect of the at least one cancer-therapeutic agent on the tumor- and tumor-adjacent normal tissues. In some cases, this may include evaluating at least one of inhibition of proliferation, induction of apoptosis, inhibition of angiogenesis, effect on vascular mimicry, status of the pathway activation, and immune-status of the tumor- and tumor-adjacent normal tissues following co-culture.

Some examples of the method and apparatus described herein may further include operations, features, means, or instructions for selecting the at least one cancer-therapeutic agent based on the at least one effect includes selecting at least one cancer therapeutic agent which induced apoptosis and/or inhibited proliferation of the tumor tissues following co-culture.

Aspects of the disclosure are initially described in the treatment of cancer in a patient, but the aspects may apply to other areas, including methods of patient-specific drug screening, selection, and/or treatment. Aspects of the disclosure are further illustrated by and described with reference to flowcharts that relate to drug screening, drug selection, and related cancer treatment methods.

### 3. BRIEF DESCRIPTION OF THE DRAWINGS

A further understanding of the nature and advantages of the present invention may be realized by reference to the following drawings. In the appended figures, similar components or features may have the same reference label. Further, various components of the same type may be distinguished by following the reference label by a dash and a second label that distinguishes among the similar components. If only the first reference label is used in the specification, the description is applicable to any one of the similar components having the same first reference label irrespective of the second reference label.

FIGS. 1A through 7D, and 8 illustrate results of multiple experiments using a method of patient-specific cancer therapy screening and related methods of treatment according to various embodiments of the invention.

FIG. 9 illustrates an example of a schematic illustrating a method of patient-specific cancer therapy screening and related methods of treatment according to various embodiments of the invention.

FIG. 10 illustrates an example of a flowchart illustrating a method of patient-specific cancer therapy screening and related methods of treatment according to various embodiments of the invention.

FIG. 11 illustrates an example of a flowchart illustrating a method of patient-specific cancer therapy screening and related methods of treatment according to various embodiments of the invention.

FIG. 12 illustrates an example of a flowchart illustrating a method of patient-specific cancer therapy screening according to various embodiments of the invention.

FIG. 13 illustrates an example of a flowchart illustrating a method of patient-specific cancer therapy screening according to various embodiments of the invention.

FIG. 14 illustrates representative results of the CTC and CAML assays according to various embodiments of the invention.

### 4. DETAILED DESCRIPTION OF THE INVENTION

This description provides examples, and is not intended to limit the scope, applicability or configuration of the invention. Rather, the ensuing description will provide those skilled in the art with an enabling description for implementing embodiments of the invention. Thus, various embodiments may omit, substitute, or add various steps and/or procedures as appropriate. For instance, it should be appreciated that the methods may be performed in an order different than that described, and that various steps may be added, omitted or combined. Also, aspects and elements described with respect to certain embodiments may be combined in various other embodiments. It should also be appreciated that the following methods, systems, and devices may individually or collectively be components of a larger system, wherein other procedures may take precedence over or otherwise modify their application.

Herein, chemotherapy or a chemotherapeutic drug may refer to one or more of anticancer drug(s), chemotherapeutic agent(s), pathway-targeted drug(s), or immune-modulatory drug(s).

Herein, tumor tissue may refer to one or more of primary cancerous tumor tissue or metastatic tumor tissue. In some cases, both tumor tissue from the primary cancer site and metastatic tumor tissue may be provided.

Traditionally it has been difficult to keep tumor tissue alive for ex-vivo testing. Methods, systems, and devices are described for ex-vivo patient-specific cancer therapy screening and related methods of treatment. In particular, methods are described herein to give a medical practitioner (e.g., without limitation, a doctor or an oncologist) an experimentally designed outcome for drug testing to assist in the selection of a treatment regimen for a patient. In some examples, a first step for screening may include culturing tumor and tumor-adjacent normal tissue with chemotherapeutic drug(s) ex-vivo. A second, optional step for screening may build off of the first step for screening. The second step may include testing tumor tissue and immune components in an ex-vivo co-culture against chemotherapeutic drug(s). In some examples, the drug(s) used in the second step may have been effective in the first step. The first and second steps allow for a patient-specific ex-vivo chemotherapeutic drug screening that may provide a more complete view of the drug effect on the tumor tissue, normal tissue, and immune system of a specific patient.

In other examples, additional optional steps may be employed to gather additional data for the medical practitioner. These include additional testing of blood obtained from the patient to identify CTCs and/or CAMLs. They may also include establishment of patient-derived (“PD”) cells (PD Cancer-Associated Fibroblast) from the tumor tissue samples and tumor-adjacent normal tissue for further testing of chemotherapy drugs and/or against genomics-guided targeted therapy drugs selected after genomic analysis of the tumor tissue samples.

In some examples, the methods provided may test different cancer treatment combinations (e.g., about one to ten drug regimens) on a patient's tumor outside of their body using ex-vivo cultures. In some methods, the ex-vivo culture of the tumor tissue may also include an immune component (e.g., CD3+ T-cells and/or CD14−/CD15+/CD16+ neutrophils) from the patient's blood to provide more data about how the patient may respond to the treatment in the presence of the patient's immune cells. Each culture type may be separated and tested independently in parallel to avoid contamination. For example, a culture may include tumor tissue, a separate culture may include tumor-adjacent normal tissue, optionally another separate culture may include tumor tissue and immune cells, optionally another separate culture may include tumor-adjacent normal tissue and immune cells, and optionally another separate culture may include tumor tissue, tumor-adjacent normal tissue, and immune cells.

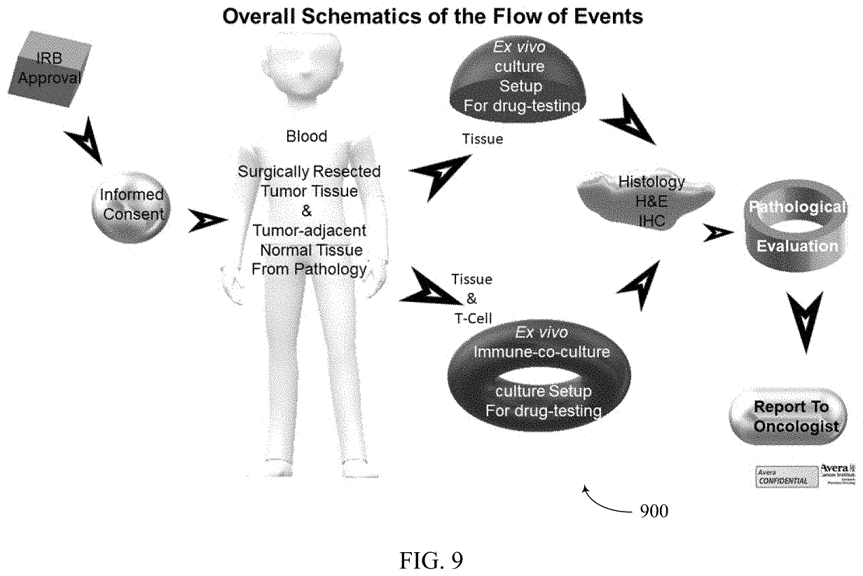

FIG. 9 illustrates the first step, optional second step, and an optional third step of process 900 that are described below at a high level and will be described in greater detail herein. The following steps may be performed by a medical professional and/or medical practitioner. In some cases, the first and second steps may be performed sequentially or side-by-side, in parallel.

First Step:

1. Obtain tissue from patient (e.g., from surgical resection and/or biopsy)
2. Decide drug(s) to test (e.g., drug(s) selected based on type of resected tissue, cancer location, cancer cell type, known genomic alterations, known signaling pathways, available clinical trial data, and/or patient history)
3. Set up ex-vivo cultures: a control culture with media and tumor tissue; a control culture with media and tumor-adjacent normal tissue; a culture with media, tumor tissue, and chemotherapeutic drug(s); a culture with media, tumor-adjacent normal tissue, and chemotherapeutic drug(s)
4. Run cultures for a predetermined time
5. Terminate cultures by fixing the tissue and optional histology
6. Perform pathological test of drug effect on the cultures
7. Report evaluation of chemotherapeutic drug(s) including the consideration of each drug-choice's effect on the patient's tumor and tumor-adjacent normal tissue to a medical practitioner

Optional Second Step:

1. Obtain tissue (e.g., from surgical resection and/or biopsy) and blood sample from patient
2. Decide immune-modulatory drug(s) to test (e.g., drug(s) selected based on patient's immune system, type of resected tissue, cancer location, cancer cell type, and/or patient history)
3. Isolate, characterize, and label immune cells from patient's blood
4. Set up ex-vivo cultures: a control co-culture with media, tumor tissue, and immune cells; and a co-culture with media, tumor tissue, immune cells, and an immune-modulatory chemotherapeutic drug
5. Run cultures for a predetermined time
6. Terminate co-cultures by fixing the tissue and optional histology
7. Perform pathological test of drug effect on the cultures
8. Report evaluation of immune-modulatory chemotherapeutic drug(s) including the consideration of each drug-choice's effect on the patient's tissue tumor and tumor-adjacent normal tissue to a medical practitioner

Optional Third Step:

1. Administer treatment to the patient using chemotherapeutic drug(s) based on the report from the first step and optionally the second step

To expand on this high level overview, an example of the first step may begin with a medical practitioner (e.g., a doctor, a surgeon, or an oncologist) surgically removing a sample of tumor tissue (e.g., ovary, omentum, endometrium, lung, breast, etc.), tumor-adjacent normal tissue (e.g., within the practitioner's margin and/or another sample of nearby normal tissue). The tumor tissue may include tissue from the primary or metastatic sites (lung, liver, lymph node, etc.). Each practitioner's margin may slightly vary as the line of control between tumor and tumor-adjacent normal tissue may be difficult to determine. Then, a medical practitioner (e.g., a doctor or pathologist) may evaluate the tissue sample (e.g., under a microscope to evaluate the anatomical and/or morphological features of the sample) to confirm what part of the sample is tumor tissue and what part is normal tissue. After removal from the patient, the tumor tissue and tumor-adjacent normal tissue may be kept separate from one another throughout the duration of the testing. This separation may extend to the machinery used during the screening process. Thus, the testing of the tissue samples may be done separately but in parallel or sequentially.

An example of the second, optional step may include a medical practitioner (e.g., a clinical nurse practitioner, a doctor, a surgeon, or an oncologist) taking a sample of blood from the patient (e.g., peripheral blood may be obtained from the same patient whose tumor is resected on the day of the surgery). The blood sample may be used to isolate immune components (e.g., leukocytes, CD3+ T-cells and/or CD14−/CD15+/CD16+ neutrophils), which are involved in the body's response to foreign substance and/or disease. A medical practitioner (e.g., a doctor or pathologist) may optionally evaluate the patient's blood (e.g., at a microscopic level from a blood smear). In some cases, the medical practitioner may check different subpopulations of leucocytes on a blood smear before the same blood may be used for the purification of CD3+-T-cells and/or CD14−/CD15+/CD16+ neutrophils and/or for different subpopulations of CD3+ T-cells and/or CD14−/CD15+/CD16+ neutrophils inside the tumor or tumor adjacent normal tissue after culture or co-culture. The leukocytes may be tested with the at least one cancer-therapeutic agent, as described herein, to determine how a patient's immune system may respond to the at least one cancer-therapeutic agent.

In some cases, this method may include a decision (e.g., logical decision) of what drug(s) to screen, for example based on the cancer type, cancer stage, cancer location, known genetic alterations, known signaling pathways, available clinical trial data, and/or a patient's medical history. In some examples, “known” may refer to information published in peer review journals. A Chemotherapeutic drug(s), targeted therapy drug(s), and/or an immune modulatory drug(s) may be picked for screening on the basis of tumor cell signaling in specific organ type tumor(s) and/or the drug history of an individual patient. The drug(s) may be tested individually or in combination. Some drugs that may be screened may include one or more of carboplatin, paclitaxel, pembrolizumab, copanlisib, everolimus, BYL719, rucaparib, olaparib, talazoparib, niraparib, trametinib, selumetinib, cobimetinib, lenvatinib, pazopinib, venetoclax, cetuximab, earlotinib, afatinib, osimertinib, ceritinib, alectinib, carfilzomib, and TAK228. More specific examples of the drug(s) that may be tested may include:

A. Standard Chemotherapy Drug(s) Dose Standardization
1. No treatment
2. Carboplatin (5 μM)+Paclitaxel 12.5 μg/ml
3. Carboplatin (10 μM)+Paclitaxel 25 μg/ml
4. Carboplatin (25 μM)+Paclitaxel 50 μg/ml
5. Carboplatin (50 μM)+Paclitaxel 75 μg/ml
6. Carboplatin (75 μM)+Paclitaxel 100 μg/ml
7. Carboplatin (25 μM)+Paclitaxel 25 μg/ml+Pembrolizumab (FDA approved immunotherapy) 10 μg/ml

B. PI3K Pathway Inhibitor (Copanlisib) Dose Standardization
1. No treatment
2. Copanlisib 20 μM
3. Copanlisib 10 μM
4. Copanlisib 5 μM
5. Copanlisib 2.5 μM
6. Copanlisib 1.0 μM
7. Copanlisib 5 μM+Pembrolizumab (FDA approved immunotherapy) 10 μg/ml
8. Everolimus (mTOR complex 1 inhibitor)
9. BYL719 (PI3K pathway inhibitor)

C. DNA Repair Pathway Inhibitor (PARP Inhibitors) Dose Standardization
1. Rucaparib
2. Olaparib
3. Talazoparib
4. Niraparib

D. MAPK Pathway Inhibitors Dose Standardization
1. Trametinib
2. Selumetinib
3. Cobimetinib

E. Angiogenic Pathway Inhibitors Dose Standardization
1. Lenvatinib
2. Pazopinib

F. Apoptotic Pathway Inhibitor
1. Venetoclax (BCL2 Inhibitor)

G. EGFR Inhibitor
1. Cetuximab (Anti-EGFR monoclonal antibody)
2. Earlotinib (EGFR kinase inhibitor)
3. Afatinib (pan ERBB family kinase inhibitor; including EGFR, HER2 and HER4)
4. Osimertinib (EGFR T790M mutation specific inhibitor)

H. ALK Fusion Inhibitor
1. Ceritinib
2. Alectinib

I. Ubiquitination Pathway Inhibitor
1. Carfilzomib

J. Combination Study (I)
1. No treatment
2. Carboplatin+Paclitaxel
3. Carboplatin+Paclitaxel+Rucaparib (PARP inhibitor, FDA approved)
4. Carboplatin+Paclitaxel+Copanlisib (PI3K inhibitor FDA approved for refractory follicular lymphoma)
5. Carboplatin+Paclitaxel+TAK228 (mTORC1/mTORC2 inhibitor, not FDA approved yet, active in clinical trial including at Avera cancer Institute)
6. Carboplatin+Paclitaxel+Trametinib (MEK1/2 inhibitor, FDA approved)
7. Pembrolizumab (FDA approved immunotherapy) 10 μg/ml+Rucaparib

K. Combination Study (II)
1. Paclitaxel+Copanlisib
2. Paclitaxel+Lenvatinib (FGFR and VEGFR inhibitor, FDA approved)
3. Paclitaxel+TAK228
4. Paclitaxel+Trametinib
5. Paclitaxel
6. Copanlisib+Pembrolizumab (FDA approved immunotherapy) 10 μg/ml

The optional T-cell and/or CD14−/CD15+/CD16+ neutrophil isolation and characterization step may include isolating leukocytes (e.g., CD3+ T-cells and/or CD14−/CD15+/CD16+ neutrophils) from a patient's peripheral blood. The leukocytes, such as CD3+ T-cells, may be characterized into multiple functional groups, for example using flow cytometry. In some cases, the groups may be characterized as CD4+, CD8+, and FOXP3+. In some examples, the leukocytes (e.g., T-cells) may be stained with a lipophilic membrane stain (e.g., Invitrogen Vybrant DiI Cell-Labeling Solution V22885) before co-culture setup as a long-term tracer to test the degree that T-cells infiltrate into the tumor. Flow cytometry analysis is performed using CD16, CD15, CD193, CD14, CD45 and CD66b (all from Miltenyi Biotec). Neutrophils were isolated by MACSxpress whole blood neutrophil Isolation kit-Miltenyi Biotec. From whole blood first we apply T-Cell isolation (described herein). The unselected cells are then run through the neutrophil isolation kit. Also a magnetic separation technique that uses an antibody cocktail obtained from Miltenyi Biotec to deplete other cells from solution selectively leaving behind the neutrophils. Residual red blood cells are lysed using ACK lysis buffer (Gibco). Miltenyi Biotec. From whole blood first T-Cell isolation is applied, as described and understood in the art. The unselected cells are then run through the neutrophil isolation kit. This is also a magnetic separation technique that uses an antibody cocktail obtained from Miltenyi Biotec to deplete other cells from a solution, selectively leaving behind the neutrophils. Residual red blood cells are lysed using ACK lysis buffer (Gibco).

An example ex-vivo culture may be set up with media and a patient's tissue sample (e.g., tumor or tumor-adjacent normal tissue). In some cases, tumor-adjacent normal tissue may be a completely different tissue type than the tumor tissue. The culture media may include one or

| | |
|---|---|
| MEDIA NAME | COMPOSITION AND/OR MODIFICATION |
| | (ROCKING AND NON-ROCKING |

more of DMEM, DMEM/F-12, and Mammary Epithelial Cell growth Media containing Glutamax, Fetal Bovine Serum, Anti-Anti (Penicillin, streptomycin, amphotericin B), Penicillin/Streptavidin, Insulin-Transferrin-Selenium (ITS), Epidermal Growth Factor, Insulin, Hydrocortisone, Bovine Pituitary Extract, Non-Essential Amino Acids, Bovine Serum Albumin, Cholera Toxin, and 5M media. Further examples of culture media that may be used in the culture set up are presented below in Table 1.

TABLE 1

| | |
|---|---|
| BASE MEDIUM 1 | DMEM + 1x Glutamax + 10% FBS + 1x Anti-Anti |
| BASE MEDIUM 2 | DMEM + 1x Glutamax + 20% FBS + 1x Anti-Anti |
| MEDIUM 1 | DMEM/F-12 + 1x Glutamax + 20% FBS + 1% P/S + 1x insulin- |
| | transferrin-selenium (ITS) |
| | Detailed composition and/or modifications during the process of |
| | standardization: |
| | DMEM:HAM's F12 = 2:1 with supplements of FCS 2%, |
| | Hydrocortisone 0.3 μg/ml, Insulin 4 μg/ml, Transferrin 4 μg/ml, |
| | 3,3′,5 Triiodothyronine 1 ng/ml, EGF 8 ng/ml, Cholera toxin |
| | 7 ng/ml, Adenine 0.2 mg/ml, Antibiotics |
| MEDIUM 2 | DMEM/F-12 + 1x Glutamax + 20% FBS + 1x P/S + Matrigel growth |
| | factor enriched |
| | Detailed composition and/or modifications during the process of |
| | standardization: |
| | RPMI-1640 with supplements of FCS 10%, E-Glutamine 2 nM, |
| | Hydrocortisone 5 μg/ml, Insulin 5 μg/ml, Cholera toxin |
| | 50 ng/ml, EGF 10 ng/ml, Antibiotics |
| MEDIUM 3 | MEGM + Additives (EGF, Insulin, Hydrocortisone, Bovine Pituitary |
| | Extract) + 10% FBS + 1% P/S |
| | Detailed composition and/or modifications during the process of |
| | standardization: |
| | RPMI-1640 supplemented with FCS 10% and antibiotics |
| MEDIUM 4 | DMEM/F-12 + 1x Glutamax + 20% FBS + 1% P/S + 1x ITS + 1x |
| | Non-Essential Amino Acids (non-EAA) |
| | Detailed composition and/or modifications during the process of |
| | standardization: |
| | DMEM:Ham's F10 = 1:1 supplemented with FCS 10% and |
| | antibiotics |
| MEDIUM 5 | DMEM/F-12 + 1x Glutamax + 20% FBS + 1% P/S + 1x ITS + 3% |
| | BSA + 1% HEPES |
| | Detailed composition &/or modifications during the process of |
| | standardization: |
| | DMEM/F-12 + Glutamax- 500 mL |
| | 20% FBS - 100 mL |
| | 1% P/S - 6 mL |
| | 1x ITS - 6 mL |
| | 3% BSA - 18.5 mL |
| | 1% HEPES - 6 mL |
| MEDIUM 5M | DMEM/F-12 + 1x Glutamax + 20% FBS + 1% P/S + 1x ITS + 3% |
| | BSA + 1% HEPES + Matrigel growth factor enriched |
| | Detailed composition &/or modifications during the process of |
| | standardization: |
| | DMEM/F-12 + Glutamax- 500 mL |
| | 20% FBS - 100 mL |
| | 1% P/S - 6 mL |
| | 1x ITS - 6 mL |
| | 3% BSA - 18.5 mL |
| | 1% HEPES - 6 mL |
| | Matrigel growth factor enriched |
| MEDIUM 6 | DMEM/F-12 + 1x Glutamax + 20% FBS + 1% P/S + 1x ITS + 1x |
| | non-EAA + 3% BSA |
| | Detailed composition &/or modifications during the process of |
| | standardization: |
| | MEBM + additives pack (EGF, hydrocortisone, bovine pituitary |
| | extract) + 10% FBS + 1% Pen/step + Matrigel growth factor enriched |
| MEDIUM 7 | MEGM + Additives (EGF, Insulin, Hydrocortisone, Bovine Pituitary |
| | Extract) + 5% FBS + 1% P/S + Cholera Toxin (25 ng/mL) |
| | Detailed composition &/or modifications during the process of |
| | standardization: |
| | MEBM - 500 mL |
| | 5% FBS - 25 mL |
| | 1% P/S - 5 mL |
| | Cholera toxin (25 ng/mL) - 6.25 μL |
| | Additives (EGF, Insulin, Hydrocortisone, Bovine Pituitary |
| | Extract) |
| MEDIUM 7M | MEGM + Additives (EGF, Insulin, Hydrocortisone, Bovine Pituitary |
| | Extract) + 5% FBS + 1% P/S + Cholera Toxin (25 ng/mL) + Matrigel |
| | growth factor enriched |
| | Detailed composition &/or modifications during the process of |
| | standardization: |
| | MEBM - 500 mL |
| | 5% FBS - 25 mL |
| | 1% P/S - 5 mL |
| | Cholera toxin (25 ng/mL) - 6.25 μL |
| | Additives (EGF, Insulin, Hydrocortisone, Bovine Pituitary |
| | Extract) |
| | Matrigel growth factor enriched |

A control culture, of either or both of tumor or tumor-adjacent normal tissue, may be maintained with no drugs. One or more screening cultures may include one or more possible chemotherapy drugs.

An example of an optional ex-vivo co-culture may be set up with media, a patient's tissue sample (e.g., tumor or tumor-adjacent normal tissue), and a patient's immune component (e.g., isolated CD3+ T-cells and/or CD14−/CD15+/CD16+ neutrophils). The culture media may include one or more of DMEM, DMEM/F-12, and Mammary Epithelial Cell growth Media containing Glutamax, Fetal Bovine Serum, Anti-Anti (Penicillin, streptomycin, amphotericin B), Penicillin/Streptavidin, Insulin-Transferrin-Selenium (ITS), Epidermal Growth Factor, Insulin, Hydrocortisone, Bovine Pituitary Extract, Non-Essential Amino Acids, Bovine Serum Albumin, Cholera Toxin, and 5M media. A control co-culture may be maintained with no chemotherapy, pathway-targeted, or immune-modulatory drugs. One or more screening co-cultures may include one or more possible treatment drugs. In some cases, co-cultured tissues with red-fluorescent dye pre-stained T-cells (whole mount and fresh frozen sections) may optionally be stained, (e.g., using H&E and/or DAPI), for the tracking and confirmation of T-cell's infiltration into the tumor in co-culture over time (e.g., at day 1 or day 2 or day 3 of the co-culture). Vital staining for neutrophil was performed to accomplish co-culture of neutrophil, T-cells and tumor tissue together. Isolated and characterized Neutrophils from patient's blood was stained using the lipophilic carbocyanine dyes DiD to obtain an uniform cellular labeling in aqueous culture media (Molecular Probes' Vybrant™ DiD cell-labeling Solution). Vybrant DiD dye delivery solution was added directly to normal culture media to uniformly label suspended neutrophils. The complementary Vybrant DiD cell-labeling solution allowed neutrophil populations to be marked in distinctive fluorescent colors (DiD [V-22887)] 644 nm and 665 nm Absorption and fluorescence Emission maxima respectively) for identification after mixing with T cells (stained with Vybrant DiL dye delivery solution) and tumor tissues during co-cultures.

In some examples, the cultures and/or co-cultures may be tested under rocking and/or non-rocking conditions. In some rocking conditions (e.g., in an incubated shaker), the culture and/or co-cultures may be rocked in 3-dimensions. The cultures and/or co-cultures may be kept at a number of different temperatures. For example, the temperature may be around 37° C. (98.6° F.). In some examples, the cultures and/or co-cultures may include a basement membrane (e.g., growth-factor enriched Matrigel). In some examples, the cultures and/or co-cultures may have a controlled exposure to CO₂. For example, if the culture and/or co-culture is in an incubator (e.g., rocking and/or non-rocking), the incubator may be maintained at, or below about 5% CO₂.

In some examples, the cultures and co-cultures may be tested in a variety of oxygen environments. In some, the cultures and co-cultures may be tested in normoxic conditions, while in others, the cultures and co-cultures may be tested in hypoxic, reoxygenation, or hyperoxic environments. In some instances, hypoxic, reoxygenation and hyperoxic conditions were created by incubating the cultures and co-cultures for hypoxic in 1% oxygen, reoxygenation at 18-20% oxygen, and hyperoxia at 40-41% oxygen concentration environments, respectively. Such conditions may be used to show the individual patient's tumor's response to oxygen. Re-oxygenation was created by bringing the tissues out of hypoxic condition to the standard culture conditions of 5% CO2. The different levels of oxygenation was maintained using an especially shield-modular incubator chamber (billups-rothenberg, CA) placed in the incubator. HIF-1alpha and VEGF will be used as the readout of hypoxia.

The cultures and/or co-cultures may be kept at the aforementioned conditions for different time durations. For example, cultures and/or co-cultures may be tested for from about 3 hours to about 90 hours, from about 6 hours to about 80 hours, or from about 12 hours to about 72 hours. In some examples, there may not be an observable change in drug efficacy after 72 hours. In some cases, the time period may be chosen to satisfy the condition that the control tumor tissue remains alive. In an example, the control tumor tissue should maintain a level of organized cellular structure, and cells should not lose their morphology. This may provide the advantage of a more precise or predicative screening process.

After the determined time period expires, the cultures and/or co-cultures may be terminated (e.g., removed from the rocker), and the remaining tissue (e.g., alive or dead) may be evaluated to determine the drug's efficacy. An evaluation of the remaining tissue may be a pathological evaluation. The evaluation may include formalin-fixing and staining tissue (e.g., with standard H&E stains and/or a panel of special IHC stains) to evaluate the drug effect on inhibition of proliferation, induction of apoptosis, inhibition of angiogenesis, evaluation of vascular mimicry, status of pathway activation, and optionally immune status of the cells in the tumor and stromal (angiogenic and immune) compartments under non-treated and drug-treated culture and/or co-culture conditions.

The evaluation of drug effect(s) based on the morphology may follow the standard H&E and IHC stains for different phenotypic markers (e.g., for the inhibition of proliferation, induction of apoptosis, inhibition of angiogenesis, evaluation of vascular mimicry, status of pathway activation, and immune-status of the tumor- and tumor-adjacent normal tissues), and the evaluation may be carried out by a medical practitioner (e.g., a certified onco-pathologist) who may be blind to the drug treatment. The evaluation (e.g., pathological evaluation) of the drug effect(s), such as the drug's efficacy of killing tumor cells, may then be considered (e.g., by a doctor or oncologist) in the determination of the patient's drug regimen. A drug regimen may then be administered based on the determination. This disclosure may provide the leverage to examine immune check-point inhibitors in combination with chemotherapy drugs in an individual patient in a time-dependent and cost-effective manner. Further, considering the uniqueness of the genomic profile of an individual tumor and the path of tumorigenic evolution in an individual patient, these methods provide a unique opportunity to evaluate drug response of tumor tissues in comparison with tumor-adjacent normal tissues in an individual patient.

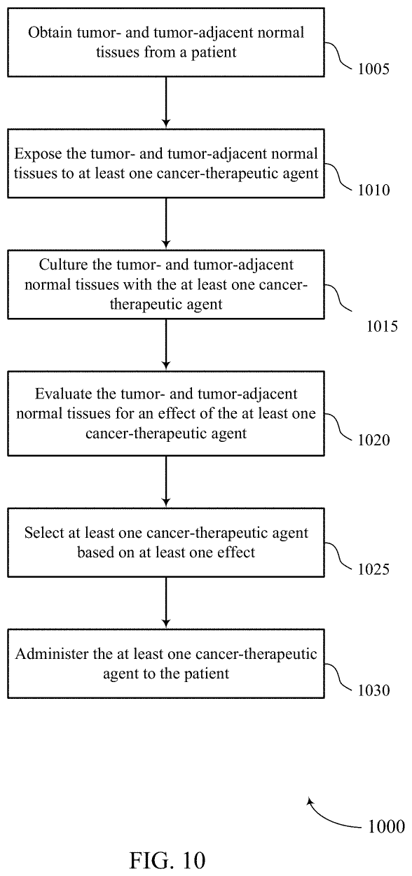

FIG. 10 shows a flowchart illustrating a method 1000 of patient-specific cancer therapy screening and related methods of treatment in accordance with aspects of the present disclosure. This exemplary method includes six steps, but more or fewer steps may be present to achieve the screening and/or treatment methods described herein.

At 1005, the method may include obtaining tumor- and tumor-adjacent normal tissues from a patient. In some examples, the tumor- and tumor-adjacent normal tissues may be obtained from the patient by surgical resection. In others, the tumor- and tumor-adjacent normal tissues may be obtained from the patient by biopsy.

At 1010, the method may include exposing the tumor- and tumor-adjacent normal tissues to at least one cancer-therapeutic agent.

At 1015, the method may include culturing the tumor- and tumor-adjacent normal tissues with the at least one cancer-therapeutic agent. In some examples, exposing the tumor- and tumor-adjacent normal tissues to at least one cancer-therapeutic agent may include culturing the tumor- and tumor-adjacent normal tissues with the at least one cancer-therapeutic agent for a period of at least about 6 hours to about 72 hours.

At 1020, the method may include evaluating the tumor- and tumor-adjacent normal tissues for an effect of the at least one cancer-therapeutic agent. Evaluating the tumor- and tumor-adjacent normal tissues for an effect of the at least one cancer-therapeutic agent on the tumor- and tumor-adjacent normal tissues may include evaluating the tumor- and tumor-adjacent normal tissues for at least one of inhibition of proliferation, induction of apoptosis, inhibition of angiogenesis, evaluation of vascular mimicry, status of pathway activation, and, and immune-status of the tumor- and tumor-adjacent normal tissues.

At 1025, the method may include selecting at least one cancer-therapeutic agent based on at least one effect of the at least one cancer-therapeutic agent on the tumor- and tumor-adjacent normal tissues. In some cases, selecting the at least one cancer-therapeutic agent based on the at least one effect of the at least one cancer-therapeutic agent on the tumor- and tumor-adjacent normal tissues may include selecting at least one cancer therapeutic agent which induced apoptosis and/or inhibited proliferation of the tumor tissues while causing little or no damage to the tumor-adjacent normal tissue. At 1030, the method may include administering the at least one cancer-therapeutic agent to the patient.

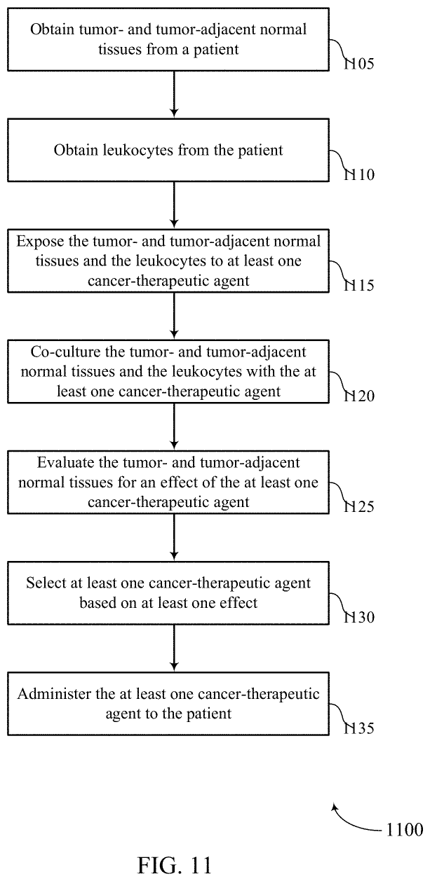

FIG. 11 shows a flowchart illustrating a method 1100 of patient-specific cancer therapy screening and related methods of treatment in accordance with aspects of the present disclosure. This exemplary method includes seven steps, but more or fewer steps may be present to achieve the screening and/or treatment methods described herein.

At 1105, the method may include obtaining tumor- and tumor-adjacent normal tissues from a patient. In some examples, the tumor- and tumor-adjacent normal tissues may be obtained from the patient by surgical resection. In others, the tumor- and tumor-adjacent normal tissues may be obtained from the patient by biopsy.

At 1110, the method may include obtaining leukocytes from the patient. In some examples, the leukocytes may be obtained from peripheral blood of the patient. The leukocytes may include CD3+ T-cells and/or CD14−/CD15+/CD16+ neutrophils from the patient. In some cases, the CD3+ T-cells and/or CD14−/CD15+/CD16+ neutrophils may be isolated from peripheral blood of the patient and used in the disclosed methods.

At 1115, the method may include exposing the tumor- and tumor-adjacent normal tissues and the leukocytes to at least one cancer-therapeutic agent in the co-culture.

At 1120, the method may include co-culturing the tumor- and tumor-adjacent normal tissues and the leukocytes with the at least one cancer-therapeutic agent. In some examples, exposing the tumor- and tumor-adjacent normal tissues to at least one cancer-therapeutic agent may include co-culturing the tumor-tissue with leukocytes and separately tumor-adjacent normal tissues with leukocytes with the at least one cancer-therapeutic agent for a period from about 6 hours to about 72 hours.

At 1125, the method may include evaluating the tumor- and tumor-adjacent normal tissues for an effect of the at least one cancer-therapeutic agent. Evaluating the tumor- and tumor-adjacent normal tissues for an effect of the at least one cancer-therapeutic agent on the tumor- and tumor-adjacent normal tissues may include evaluating the tumor- and tumor-adjacent normal tissues for at least one of inhibition of proliferation, induction of apoptosis, inhibition of angiogenesis, evaluation of vascular mimicry, status of pathway activation, and, and immune-status of the tumor- and tumor-adjacent normal tissues.

At 1130, the method may include selecting at least one cancer-therapeutic agent based on at least one effect of the at least one cancer-therapeutic agent on the tumor- and tumor-adjacent normal tissues. In some cases, selecting the at least one cancer-therapeutic agent based on the at least one effect of the at least one cancer-therapeutic agent on the tumor- and tumor-adjacent normal tissues may include selecting at least one cancer therapeutic agent which induced apoptosis and/or inhibited proliferation of the tumor tissues while causing little or no damage to the tumor-adjacent normal tissue. At 1135, the method may include administering the at least one cancer-therapeutic agent to the patient.

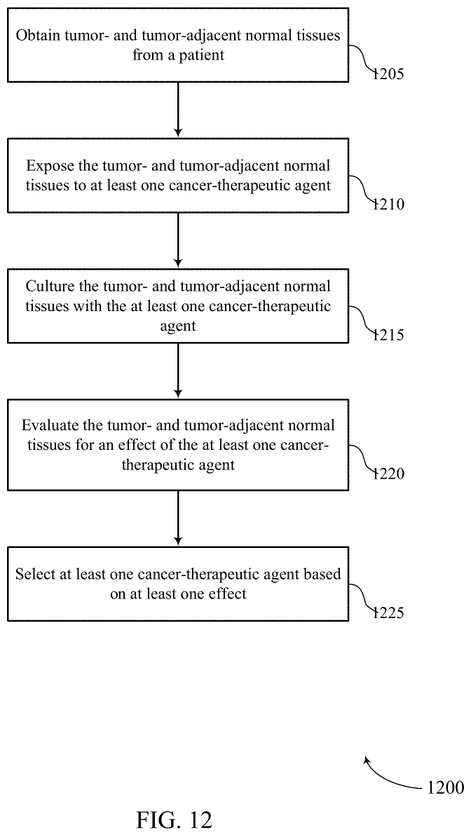

FIG. 12 shows a flowchart illustrating a method 1200 of patient-specific cancer therapy screening in accordance with aspects of the present disclosure. This exemplary method includes five steps, but more or fewer steps may be present to achieve the methods of patient-specific evaluation of cancer-therapeutic agents described herein.

At 1205, the method may include obtaining tumor- and tumor-adjacent normal tissues from a patient. In some examples, the tumor- and tumor-adjacent normal tissues may be obtained from the patient by surgical resection. In others, the tumor- and tumor-adjacent normal tissues may be obtained from the patient by biopsy. The tumor- and tumor-adjacent normal tissues may be resected and/or biopsied from the margin of a tumor of the patient or from separate sites as determined by a medical practitioner.

At 1210, the method may include exposing the tumor- and tumor-adjacent normal tissues to at least one cancer-therapeutic agent.

At 1215, the method may include culturing the tumor- and tumor-adjacent normal tissues with the at least one cancer-therapeutic agent. In some examples, exposing the tumor- and tumor-adjacent normal tissues to at least one cancer-therapeutic agent may include culturing the tumor- and tumor-adjacent normal tissues with the at least one cancer-therapeutic agent for a period of at least about 6 hours to about 72 hours.

At 1220, the method may include evaluating the tumor- and tumor-adjacent normal tissues for an effect of the at least one cancer-therapeutic agent. Evaluating the tumor- and tumor-adjacent normal tissues for an effect of the at least one cancer-therapeutic agent on the tumor- and tumor-adjacent normal tissues may include evaluating the tumor- and tumor-adjacent normal tissues for at least one of inhibition of proliferation, induction of apoptosis, inhibition of angiogenesis, evaluation of vascular mimicry, status of pathway activation, and, and immune-status of the tumor- and tumor-adjacent normal tissues.

At 1225, the method may include selecting at least one cancer-therapeutic agent based on at least one effect of the at least one cancer-therapeutic agent on the tumor- and tumor-adjacent normal tissues. In some cases, selecting the at least one cancer-therapeutic agent based on the at least one effect of the at least one cancer-therapeutic agent on the tumor- and tumor-adjacent normal tissues may include selecting at least one cancer therapeutic agent which induced apoptosis and/or inhibited proliferation of the tumor tissues while causing little or no damage to the tumor-adjacent normal tissue.

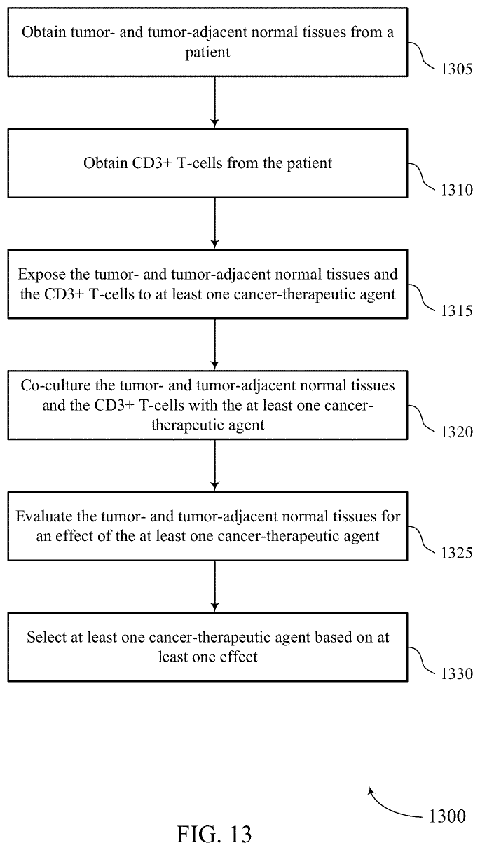

FIG. 13 shows a flowchart illustrating a method 1300 of patient-specific cancer therapy screening in accordance with aspects of the present disclosure. This exemplary method includes six steps, but more or fewer steps may be present to achieve the methods of patient-specific evaluation of cancer-therapeutic agents described herein.

At 1305, the method may include obtaining tumor- and tumor-adjacent normal tissues from a patient. In some examples, the tumor- and tumor-adjacent normal tissues may be obtained from the patient by surgical resection. In others, the tumor- and tumor-adjacent normal tissues may be obtained from the patient by biopsy. The tumor- and tumor-adjacent normal tissues may be resected and/or biopsied from the margin of a tumor of the patient.

At 1310, the method may include obtaining CD3+ T-cells and/or CD14−/CD15+/CD16+ neutrophils from the patient. In some examples, CD3+ T-cells and/or CD14−/CD15+/CD16+ neutrophils may be obtained from blood from the patient, for example, from a peripheral blood sample from the patient. In some cases, CD3+ T-cells and/or CD14−/CD15+/CD16+ neutrophils may be obtained from the blood from the patient by isolation using magnetic beads.

At 1315, the method may include exposing the tumor- and tumor-adjacent normal tissues and the CD3+ T-cells and/or CD14−/CD15+/CD16+ neutrophils to at least one cancer-therapeutic agent in the co-culture. In some examples, the at least one cancer-therapeutic agent may be one or more of a chemotherapeutic agent, a pathway-targeted drug, or an immune-modulatory drug. In the co-culture with CD3+ T-cells and/or CD14−/CD15+/CD16+ neutrophils, an immune-modulatory drug may be tested, alone or in combination with chemotherapy and/or pathway targeted drugs.

At 1320, the method may include co-culturing the tumor- and tumor-adjacent normal tissues and the CD3+ T-cells and/or CD14−/CD15+/CD16+ neutrophils with the at least one cancer-therapeutic agent. In some examples, exposing the tumor- and tumor-adjacent normal tissues to at least one cancer-therapeutic agent may include co-culturing the tumor-tissue with CD3+ T-cells and/or CD14−/CD15+/CD16+ neutrophils and separately tumor-adjacent normal tissues with CD3+ T-cells and/or CD14−/CD15+/CD16+ neutrophils with the at least one cancer-therapeutic agent (e.g., immune-modulatory drug) for a period of at least about 6 hours to about 72 hours.

At 1325, the method may include evaluating the tumor- and tumor-adjacent normal tissues for an effect of the at least one cancer-therapeutic agent. Evaluating the tumor- and tumor-adjacent normal tissues for an effect of the at least one cancer-therapeutic agent on the tumor- and tumor-adjacent normal tissues may include evaluating the tumor- and tumor-adjacent normal tissues for at least one of inhibition of proliferation, induction of apoptosis, inhibition of angiogenesis, evaluation of vascular mimicry, status of pathway activation, and, and immune-status of the tumor- and tumor-adjacent normal tissues.

At 1330, the method may include selecting at least one cancer-therapeutic agent based on at least one effect of the at least one cancer-therapeutic agent on the tumor- and tumor-adjacent normal tissues. In some cases, selecting the at least one cancer-therapeutic agent based on the at least one effect of the at least one cancer-therapeutic agent on the tumor- and tumor-adjacent normal tissues may include selecting at least one cancer therapeutic agent which induced apoptosis and/or inhibited proliferation of the tumor tissues while causing little or no damage to the tumor-adjacent normal tissue.

The methods of FIGS. 10-14 may each include any one or more of several additional steps. In one, an additional evaluation is made of the blood sample taken from the patient. The sample is assayed for circulating tumor cells (“CTCs”), which may include tumor cells sloughed off from the primary tumor site which circulate throughout the body through the lymphatic system and blood vessels, which may, in some instances, colonize new places. CTC is a marker/indicator for the state of aggressiveness of the tumor and also is directly related to the outcome/progression of the disease. The presence of CTCs may serve as an indicator that there is a risk of secondary tumors, and may serve to inform a medical professional of the possibility of such tumors, and provide additional data valuable for making a longer-term treatment proposal for future management/monitoring of the disease. In some instances, this step may be repeated with new blood samples from the patient at intervals following initial treatment (longitudinal study). In some instances, the test would be repeated at 3-month intervals. In others, it would be repeated at 6-month intervals. Other intervals may be prescribed by the medical professional, as well.

Another optional additional step is a further evaluation of the blood sample taken from the patient. The sample is assayed for circulating cancer associated macrophage-like cells (“CAMLs”) the size and number of CAMLs has been shown to be negatively related to clinical outcome. In some instances, CAMLs have been shown to be more prevalent than CTCs, which may be elusive. Further, CAMLs have been shown to be present in 95% of all stages of cancer patients, but not present in normal subjects. This step of assaying for CAMLs may be done prior to chemotherapy or surgical resection of a tumor, and then the test may be repeated again to monitor for changes following the procedure. Monitoring may be done for absence, presence, number and size of CAMLs. In some instances, this optional test may be done in concert with testing for CTCs, and in some instances from the same blood sample to provide more relevant clinical information to oncologists. Indeed, in some instances, no additional blood sample would be required for the evaluation of CAML. Several methods have been used for identification of CTCs and CAMLs. One of the methods used a commercially available kit (Microfiltration followed by specific marker-based triple immune-fluorescence (CellSieve™ CTC Enumeration kit-Creatv Microtech)). Other identification procedures include (1) Thin-Prep followed by DiffQuick, (2) Thin-Prep followed by specific marker-based double-ICC; (3) microfiltration followed by specific marker-based double-ICC, (4) Parallel staining of Microfiltration followed by specific marker-based triple immune-fluorescence and Thin-Prep followed by specific marker-based double-ICC, and (5) Parallel staining of Microfiltration followed by specific marker-based triple immune-fluorescence and Microfiltration followed by specific marker-based double-ICC. In these methods, the microfiltration followed by specific marker-based double-ICC step may include the use of any one or more of the following: high molecular weight cytokeratin (cytokeratin 8, 18), EpCAM, EphB4, CEA, CD14, CD45, CD68, and/or CD16. The number of methods is employed because CTCs may be found in only very low numbers in patients' blood and are thus known be to elusive. CTCs may be identified and characterized using methods and kits commercially available from CellSieve.

We employed several methods of Identification of CTC-CAML because CTCs are very low in numbers in patients' blood (7.5 ml) and thus known be to elusive (Published fact). Hence we designed several methods to independently substantiate our finding (by the commercially available method). Testing/determining a particular item by several independent methods mathematically decreases the probability of “error” in the detection system.

Another optional additional step is the genetic analysis of the tissue samples taken from the patient. The sample is assayed for known cancer biomarkers and genetic alterations to establish a treatment regimen based on the genetic identification of the cancer. This genomic-guided personalized medicine approach can then be tested against at least a portion of the tissue sample. Since this process of genetic analysis typically requires more time for completion than the ex vivo culture as well as ex vivo co-culturing steps disclosed (which may be completed within 72 hours of the acquisition of the tissue/biopsy samples), separately and in some instances, in parallel to the co-culture steps, a portion of the tumor tissue sample is cultured to prepare patient-derived cells. These cells are cultured for a longer period than the ex vivo culture as well as ex vivo co-culturing steps steps to allow testing of the sample against the treatment(s) recommended by both the ex vivo culture as well as ex vivo co-culturing steps steps taught herein and/or the treatment recommended by the outcome of the genomic testing. In some instances, the portion of the tumor tissue sample is cultured over the selected time to selectively preserve Cancer Associated Fibroblasts from the tumor sample. A portion of the tumor tissue sample is cultured as Patient-Derived Cells which was cultured without enzymatic-digestion from both tumor-adjacent normal tissue and tumor tissue, separately. Thus Cancer-Associated Fibroblasts (from tumor tissue) and normal fibroblasts (from normal tissue) were cultured over the selected time through passages to selectively preserve the Cancer-Associated Fibroblasts for future drug-testing.

The reactions of the Cancer Associated Fibroblasts may be indicative of the reaction of the tumor tissue generally to the prescribed therapeutic regimen. More specifically, if the cultured Cancer Associated Fibroblasts die in response to the treatment, it would be expected that the drug-combination will have a direct influence on the tumor cells. This allows comparison of the ex vivo culture as well as ex vivo co-culturing treatment regimen results with the results of the treatment prescribed by genomically-derived testing, providing more and more relevant information to the oncologist.

Ex vivo culture of Patient Derived cells from tumor and tumor-adjacent normal tissue is done from the “feeder matrix” without any enzymatic digestion. A “feeder matrix” is created from the original tumor tissue and the tumor-adjacent normal tissue. A “feeder matrix” contains all the cellular content of the original tissue received from the surgery/biopsy. The culture is set up on cover-slips using ex vivo culture media. The cells growing on the cover-slips are successively passaged by differential-trypsinization and selective-adhesion techniques to obtain fibroblasts. Alpha smooth muscle antigen (aSMA), vimentin and Fibroblast Associated Protein (FAP) will be used as a marker.

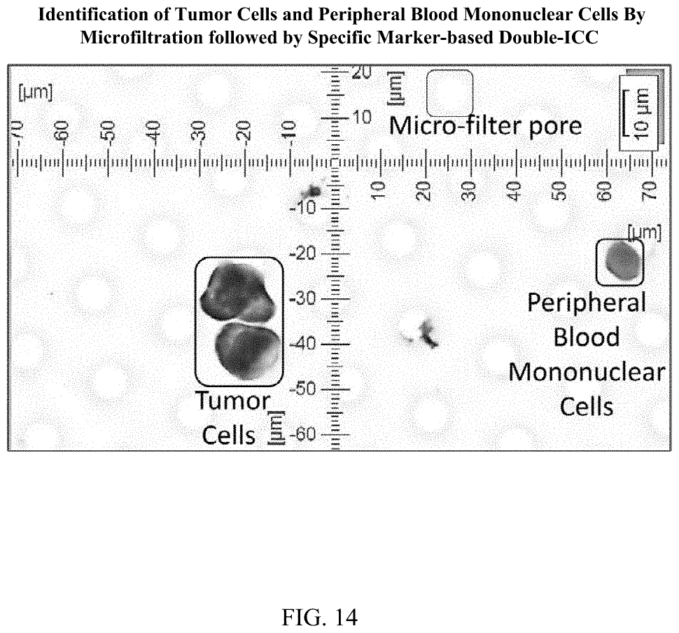

FIG. 14 illustrates representative results of the CTC and CAML assays according to various embodiments of the invention. Specifically, FIG. 14 shows exemplary tumor and peripheral blood mononuclear cells on a microfilter, stained for visualization as described herein. In this image, NCI-H441 lung cancer cells and peripheral blood mononuclear cells were used as representative of CTC cells and CAMLs, respectively. Figure Legend: Using a tumor cell (NCI-H441, a lung cancer cell line) and PBMC (Peripheral Blood Mononuclear Cells) we have developed an independent method of identifying CTC (as represented by tumor cell) and CAML (as represented by PBMC).

It should be noted that the methods described herein describe possible implementations, and that the operations and the steps may be rearranged or otherwise modified and that other implementations are possible. Further, aspects from two or more of the methods may be combined.

### 5. EXAMPLES OF THE INVENTION

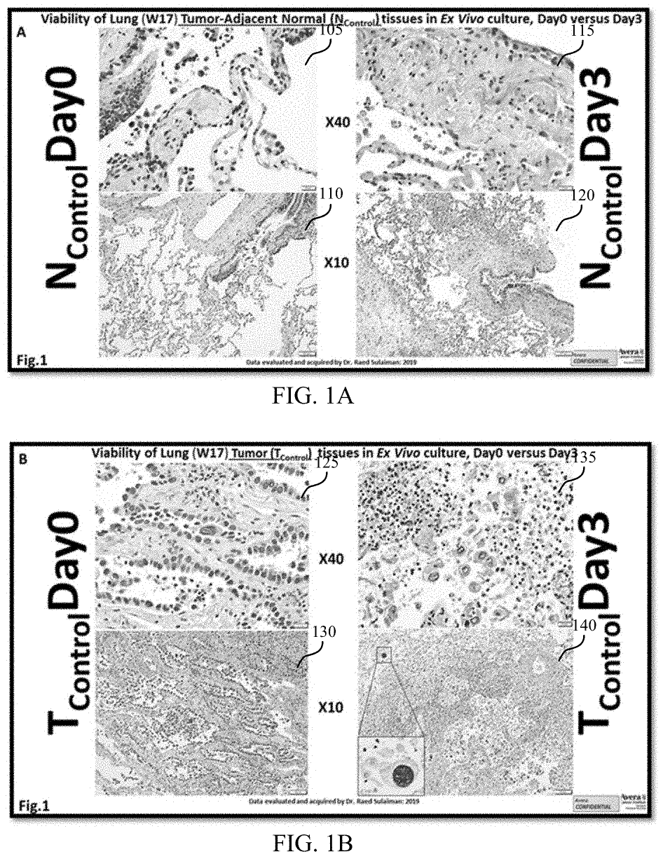

### Example 1—Viability of Tumor (T_{Control}) and Tumor-Adjacent Normal (N_{Control}) Tissues in Ex-Vivo Culture of Adenocarcinoma of the Lung as Presented in the FIG. 1: Patient #W17

Method: Resected samples (IRB approved) of the tumor and tumor-adjacent normal tissues were received from a patient with adenocarcinoma of the lung (pT2aN0). The samples were grossed by a certified Pathologist. Tumor and tumor-adjacent normal tissues were separately cultured ex-vivo in commercially available artificial basement membrane (Matrigel procured from BD BioSciences, USA) in media 5M (as described in Table 1) for 3 days under 5% CO2 at 37 degrees Centigrade. The culture was terminated at 24 hours, 48 hours, and 72 hours by fixation in formaldehyde. The fixed tissues were processed for routine histology. The viability of tissues in 3 days of ex-vivo culture was evaluated by a certified Pathologist in the H&E stained tissue sections shown in FIGS. 1A and 1B.

Media: The 5M media was used and contains DMEM/F-12, 1× Glutamax, 20% FBS, 1% P/S, 1×ITS, 3% BSA, 1% HEPES, and Matrigel growth factor enriched.

Drug(s): No drug was used for the validation of the tissue viability in ex-vivo culture.

Results: FIGS. 1A and 1B show the results from this example.

As illustrated in FIGS. 1A and 1B, the tissues were viable in ex-vivo culture. Both Tumor-Adjacent Normal (N_{Control}) tissues as shown in FIG. 1A and Tumor (T_{Control}) tissues as shown in FIG. 1B are viable after ex-vivo cultures for Day 1, Day 2, and Day 3 as compared to non-cultured tissue, Day 0. Representative pictures of only Day 3 are provided. As shown in FIG. 1A, the photomicrograph shows the preservation of tissue elements including alveolar, bronchiolar, and vascular tissue with the supporting stroma. Higher magnification shows the preservation of cellular elements including intact cilia, RBC, endothelial cells and macrophages on day 0, shown by photomicrographs 105 and 110. The photomicrograph on Day 3 shows preservation of tissue elements including alveolar, bronchiolar, and vascular tissue with the supporting stroma. Higher magnification shows the preservation of cellular elements including intact cilia, RBC, endothelial cells and macrophages in ex-vivo culture, shown by photomicrographs 115 and 120. Preservation of cellular components and features on Day 3 are comparable to Day 0. As shown in FIG. 1B, the photomicrographs 125 and 130 show preservation of tissue elements including T-cells and other lung stromal components at day 0. On Day 3 of ex-vivo culture, preservation of tumor and non-tumor cellular element, pigments and macrophages are observed in photomicrographs 135 and 140. A large viable tumor cell is shown in the box of photomicrograph 140.

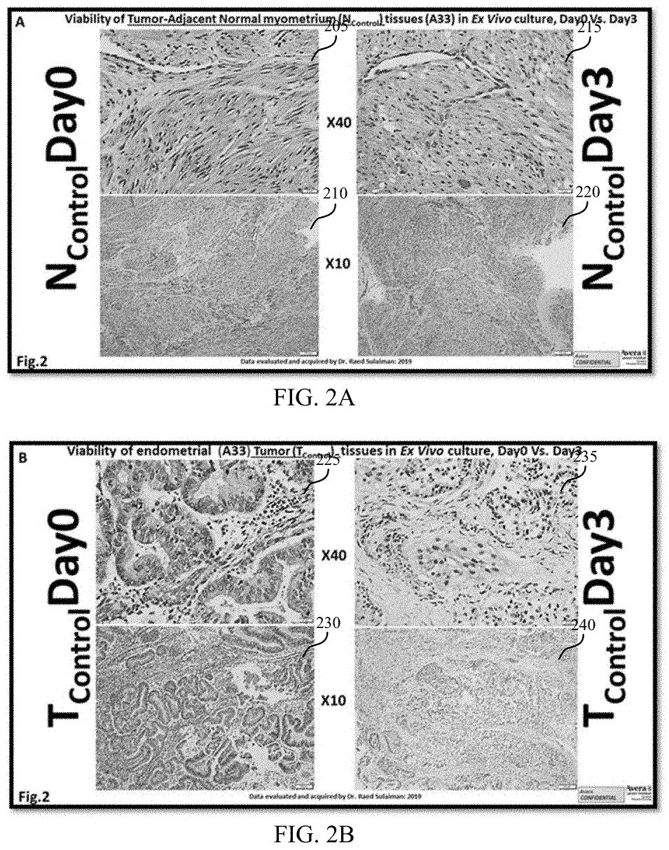

### Example 2—Viability of Tumor (T_{Control}) and Tumor-Adjacent Normal (N_{Control}) Tissues in Ex-Vivo Culture of Adenocarcinoma of Endometrium (Endometrioid Type) as Presented in the FIG. 2: Patient #A33

Method: Resected samples (IRB approved) of the tumor and tumor-adjacent normal tissues were received from a patient with adenocarcinoma of the endometrium (pT1aN0). The samples were grossed by a certified Pathologist. Tumor and tumor-adjacent normal tissues were separately cultured ex-vivo in commercially available artificial basement membrane (Matrigel procured from BD BioSciences, USA) in media 5M (as described in Table 1) for 3 days under 5% CO2 at 37 degrees Centigrade. The culture was terminated at 24 hours, 48 hours, and 72 hours by fixation in formaldehyde. The fixed tissues were processed for routine histology. The viability of tissues in 3 days of ex-vivo culture was evaluated by a certified Pathologist in the H&E stained tissue sections shown in FIGS. 2A and 2B.

Media: The 5M media was used and contains DMEM/F-12, 1× Glutamax, 20% FBS, 1% P/S, 1×ITS, 3% BSA, 1% HEPES, and Matrigel growth factor enriched.

Drug(s): No drug was used for the validation of the tissue viability in ex-vivo culture.

Results: FIGS. 2A and 2B show the results from this example.

As shown in FIGS. 2A and 2B, the tissues were viable in ex-vivo culture. Both Tumor-Adjacent Normal (N_{Control}) myometrial tissues as shown in FIG. 2A and endometrial Tumor (T_{Control}) tissues as shown in FIG. 2B are viable after ex-vivo cultures for Day 1, Day 2, and Day 3 as compared to non-cultured tissue, Day 0. Representative pictures of only Day 3 are provided. As shown in FIG. 2A, the photomicrograph shows the preservation of tissue elements of myometrium with supporting stroma and capillaries. Higher magnification shows the preservation of cellular elements on day 0 shown by photomicrographs 205 and 210. The photomicrograph on Day 3 shows preservation of tissue elements including the supporting stroma. Higher magnification shows the preservation of cellular elements in ex-vivo culture shown by photomicrographs 215 and 220. Preservation of cellular components and features of the tumor-adjacent normal tissue (myometrium) on Day 3 are comparable to Day 0. As shown in FIG. 2B, the photomicrograph shows the preservation of tumor proliferation and stromal components on day 0. Preserved tumor epithelial cells and benign stromal components are visible in the photomicrographs 225 and 230. On Day 3 of ex-vivo culture, preservation of tumor and non-tumor cellular element are observed in photomicrographs 235 and 240. Stromal and epithelial components are preserved.

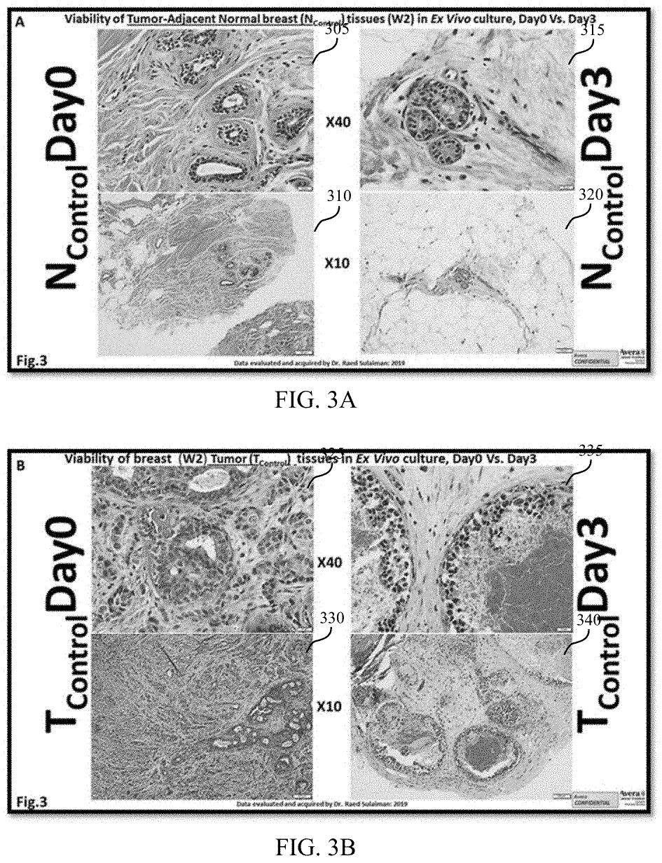

### Example 3—Viability of Tumor (T_{Control}) and Tumor-Adjacent Normal (N_{Control}) Tissues in Ex-Vivo Culture of Invasive Ductal Carcinoma of Breast as Presented in the FIG. 3: Patient #W2

Method: Resected samples (IRB approved) of the tumor and tumor-adjacent normal tissues were received from a patient with Invasive Ductal Carcinoma of the breast (Nottingham grade II). The samples were grossed by a certified Pathologist. Tumor and tumor-adjacent normal tissues were separately cultured ex-vivo in commercially available artificial basement membrane (Matrigel procured from BD BioSciences, USA) in media 1M (as described in Table 1) for 3 days under 5% CO2 at 37 degrees Centigrade. The culture was terminated at 24 hours, 48 hours, and 72 hours by fixation in formaldehyde. The fixed tissues were processed for routine histology. The viability of tissues in 3 days of ex-vivo culture was evaluated by a certified Pathologist in the H&E stained tissue sections shown in FIGS. 3A and 3B.

Media: The 1M media was used and contains DMEM/F-12, 1× Glutamax, 20% FBS, 1% P/S, and 1× insulin-transferrin-selenium (ITS).

Drug(s): No drug was used for the validation of the tissue viability in ex-vivo culture.

Results: FIGS. 3A and 3B show the results from this example.

As shown in FIGS. 3A and 3B, the tissues were viable in ex-vivo culture. Both Tumor-Adjacent Normal (N_{Control}) breast tissues as shown in FIG. 3A and breast Tumor (T_{Control}) tissues as shown in FIG. 3B are viable after ex-vivo cultures for Day 1, Day 2, and Day 3 as compared to non-cultured tissue, Day 0. Representative pictures of only Day 3 are provided. As shown in FIG. 3A, the photomicrograph shows the preservation of tissue elements of the normal breast with fibro-fatty tissue with breast ductal epithelium. Higher magnification shows the preservation of cellular elements on day 0 shown by photomicrographs 305 and 310. The photomicrograph on Day 3 shows preservation of tissue elements including the supporting stroma. Higher magnification shows the preservation of cellular elements in ex-vivo culture shown by photomicrographs 315 and 320. Preservation of cellular components and features of the tumor-adjacent normal breast tissue on Day 3 are comparable to Day 0. As shown in FIG. 3B, the photomicrograph shows preservation of in situ tissue on day 0. Preserved tumor epithelial cells and benign stromal components are visible in the photomicrographs 325 and 330. On Day 3 of ex-vivo culture, preservation of tumor and non-tumor cellular element showing some degree of the aging process are observed in the photomicrographs 335 and 340.

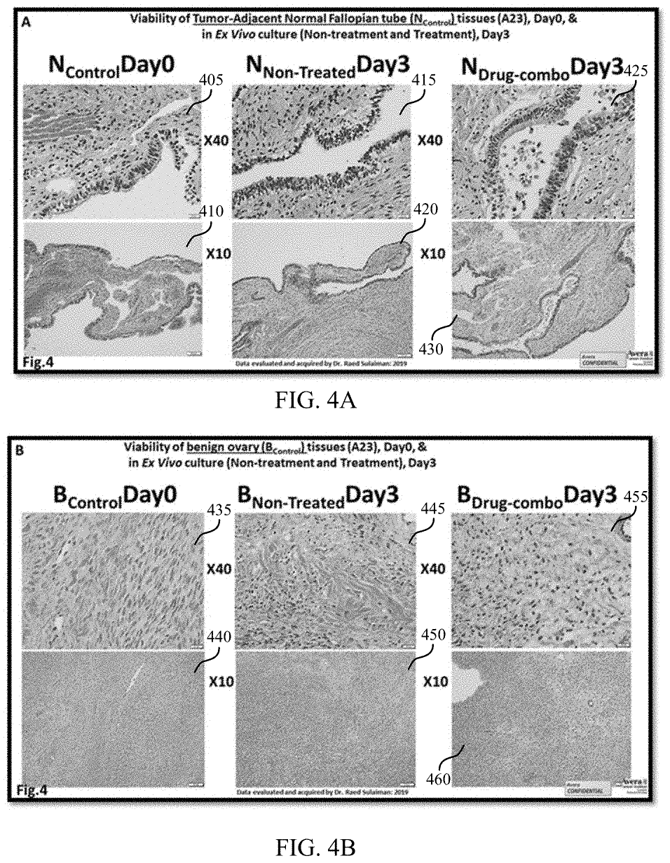

### Example 4—Drug Combination(s) Tested on Internal Control Tissue of Fallopian Tube in Ex-Vivo Culture as Presented in the FIG. 4: Patient #A23

Method: Resected samples (IRB approved) of the tumor and tumor-adjacent normal tissues were received from a patient with the tumor of the Fallopian tube. The samples were grossed by a certified Pathologist. Tumor and tumor-adjacent normal tissues were separately cultured ex-vivo in commercially available artificial basement membrane (Matrigel procured from BD BioSciences, USA) in media 5M (as described in Table 1) for 3 days under 5% CO2 at 37 degrees Centigrade. The culture was terminated at 24 hours, 48 hours, and 72 hours by fixation in formaldehyde. The fixed tissues were processed for routine histology. The viability of tissues in 3 days of ex-vivo culture was evaluated by a certified Pathologist in the H&E stained tissue sections shown in FIGS. 4A and 4B.

Media: The 5M media was used and contains DMEM/F-12, 1× Glutamax, 20% FBS, 1% P/S, 1×ITS, 3% BSA, 1% HEPES, and Matrigel growth factor enriched.

Drug(s): Five drug combinations (carboplatin and paclitaxel; carboplatin, paclitaxel, and everolimus; carboplatin, paclitaxel, and lenvatenib; carboplatin, paclitaxel, and rucaparib; carboplatin, paclitaxel, and trametinib) were used for the validation of the tissue viability in ex-vivo culture.

Results: FIGS. 4A and 4B show the results from this example.

As shown in FIGS. 4A and 4B, the tissues were viable in ex-vivo culture. As shown in FIG. 4A, the photomicrograph shows preservation of normal tissue elements of the Fallopian tube. Higher magnification shows the preservation of cellular elements on day 0 shown by photomicrographs 405 and 410. The photomicrograph of non-treated (NT) normal tissue 415 and 420, and treated (drug-combo) normal tissue 425 and 430 on Day 3 shows that there is no effect of the drug. As shown in FIG. 4B, the photomicrograph shows the preservation of tissue elements. Higher magnification shows the preservation of cellular elements on day 0 shown by photomicrographs 435 and 440. The photomicrograph of non-treated (NT) benign tissue 445 and 450 and treated (drug-combo) benign tissue 455 and 460 on Day 3 shows that there is no effect of the drug.

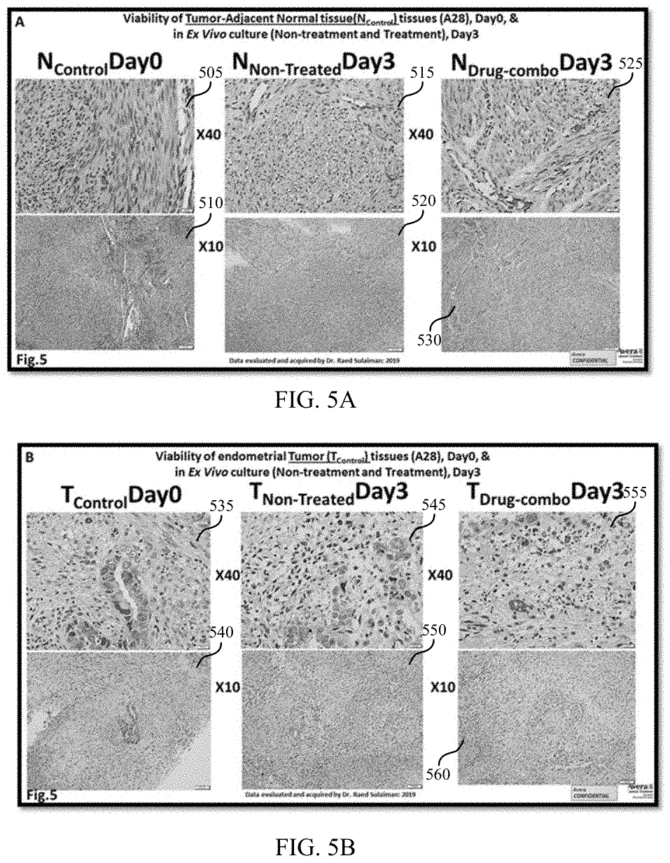

### Example 5—Drug Induced Apoptosis in Ex-Vivo Culture of Tumor Tissues and Tumor-Adjacent Normal Tissues in Carcinosarcoma of Endometrium as Presented in the FIG. 5: Patient #A28

Method: Resected samples (IRB approved) of the tumor and tumor-adjacent normal tissues were received from a patient with carcinosarcoma of the endometrium (pT1aN0). The samples were grossed by a certified Pathologist. Tumor and tumor-adjacent normal tissues were separately cultured ex-vivo in commercially available artificial basement membrane (Matrigel from BD BioSciences, USA) in media 5M (as described in Table 1) for 3 days under 5% CO2 at 37 degrees Centigrade. The culture was terminated at 24 hours, 48 hours, and 72 hours by fixation in formaldehyde. The fixed tissues were processed for routine histology. Apoptosis in tissues was evaluated by a certified Pathologist in the H&E stained tissue sections shown in FIGS. 5A and 5B.

Media: The 5M media was used and contains DMEM/F-12, 1× Glutamax, 20% FBS, 1% P/S, 1×ITS, 3% BSA, 1% HEPES, and Matrigel growth factor enriched.

Drug(s): Five drug combinations (paclitaxel; paclitaxel and BKM120; paclitaxel and lenvatenib; paclitaxel and TAK228; paclitaxel and trametinib) were used for testing the drug-induced apoptosis in the tissues in ex-vivo culture.

Results: FIGS. 5A and 5B show the results from this example.

Drug-induced apoptosis in ex-vivo culture are presented in FIGS. 5A and 5B. Drug combination(s) tested on both endometrial carcinosarcoma Tumor (T_{Drug-Combo}) and Tumor-Adjacent Normal (N_{Drug-Combo}) tissues in ex-vivo cultures for Day 1, Day 2, and Day 3 as compared to non-treated tissue (NT) on corresponding days. Drug-induced apoptosis has been morphologically identified in tumor tissues, Tumor (T_{Drug-Combo}) as nuclear-debris in contrast to Tumor Day 0 (TD0), as well as non-treated (NT) Tumor control (T_{Control}). No change was observed in Tumor-Adjacent Normal tissues following drug treatment (N_{Drug-Combo}) as compared to its corresponding control (N_{Control}). Tumor-adjacent normal (N_{Control}) tissues as shown in photomicrographs 515 and 520 and drug treatment (N_{Drug-Combo}) as shown in photomicrographs 525 and 530 are viable in ex-vivo cultures for Day 1, Day 2, and Day 3 as compared to non-cultured tissues (ND0) as shown in photomicrographs 505 and 510. Representative pictures of the only Day 3 with or without drug are provided. Tumor (T_{Control}) tissues are shown in photomicrographs 545 and 550 and drug treatment (T_{Drug-Combo}) are shown in photomicrographs 555 and 560. Drug-induced apoptotic changes were observed in photomicrographs 555 and 560 in ex-vivo cultures for Day 1, Day 2, and Day 3 as compared to non-treated tissues as shown in photomicrographs 545 and 550, respectively. Representative pictures of the only Day 3 with or without drug are provided. Tumor (T_{Control}) (NT) tissues are found viable in ex-vivo cultures for Day 1, Day 2, and Day 3 as compared to non-cultured tissues (TD0) as shown in photomicrographs 535 and 540.

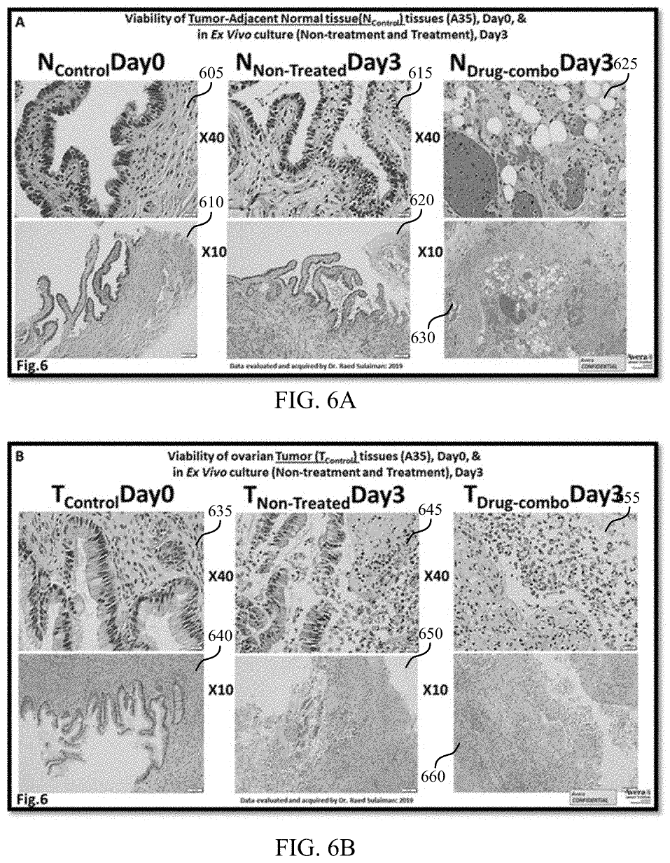

### Example 6—Drug Induced Apoptosis in Ex-Vivo Culture of Tumor Tissues and Tumor-Adjacent Normal Tissues in Ovarian Tumor as Presented in the FIG. 6: Patient #A35

Method: Resected samples (IRB approved) of the tumor and tumor-adjacent normal tissues were received from a patient with ovarian tumor (pT1N0). The samples were grossed by a certified Pathologist. Tumor and tumor-adjacent normal tissues were separately cultured ex-vivo in commercially available artificial basement membrane (Matrigel from BD BioSciences, USA) in media 5M (as described in Table 1) for 3 days under 5% CO2 at 37 degrees Centigrade. The culture was terminated at 24 hours, 48 hours, and 72 hours by fixation in formaldehyde. The fixed tissues were processed for routine histology. Apoptosis in tissues was evaluated by a certified Pathologist in the H&E stained tissue sections shown in FIGS. 6A and 6B.

Media: The 5M media contains DMEM/F-12, 1× Glutamax, 20% FBS, 1% P/S, 1×ITS, 3% BSA, 1% HEPES, and Matrigel growth factor enriched.

Drug(s): Five drug combinations (carboplatin and paclitaxel; carboplatin, paclitaxel, and rucaparib; carboplatin, paclitaxel, and copanlisib; carboplatin, paclitaxel, and TAK228; carboplatin, paclitaxel, and lenvatenib) were used for testing the drug-induced apoptosis in the tissues in ex-vivo culture.

Results: FIGS. 6A and 6B show the results from this example.

Drug-induced apoptosis in ex-vivo culture are presented in FIGS. 6A and 6B. Drug combination(s) tested on both ovarian Tumor (T_{Drug-Combo}) and Tumor-Adjacent Normal (N_{Drug-Combo}) tissues in ex-vivo cultures for Day 1, Day 2, and Day 3 as compared to non-treated (NT) tissue on corresponding days. Drug-induced apoptosis has been morphologically identified in tumor tissues, Tumor (T_{Drug-Combo}) as nuclear-debris in contrast to Tumor Day 0 (TD0), as well as non-treated Tumor control (T_{Control}). No change was observed in Tumor-Adjacent Normal tissues following drug treatment (N_{Drug-Combo}) as compared to its corresponding control (N_{Control}). Tumor-adjacent normal (N_{Control}) tissues as shown in photomicrographs 615 and 620 and drug treatment (N_{Drug-Combo}) as shown in photomicrographs 625 and 630 are viable in ex-vivo cultures for Day 1, Day 2, and Day 3 as compared to non-cultured tissues (ND0) as shown in photomicrographs 605 and 610. Representative pictures of the only Day 3 with or without drug are provided. Tumor (T_{Control}) tissues are shown in photomicrographs 645 and 650 and drug treatment (T_{Drug-Combo}) are shown in photomicrographs 655 and 660. Drug-induced apoptotic changes were observed in photomicrographs 655 and 660 in ex-vivo cultures for Day 1, Day 2, and Day 3 as compared to non-treated tissues as shown in photomicrographs 645 and 650. Representative pictures of the only Day3 with or without drug are provided. Tumor (T_{Control}) tissues (NT) are found viable in ex-vivo cultures for Day 1, Day 2, and Day 3 as compared to non-cultured tissues (TD0) as shown in photomicrographs 635 and 640.

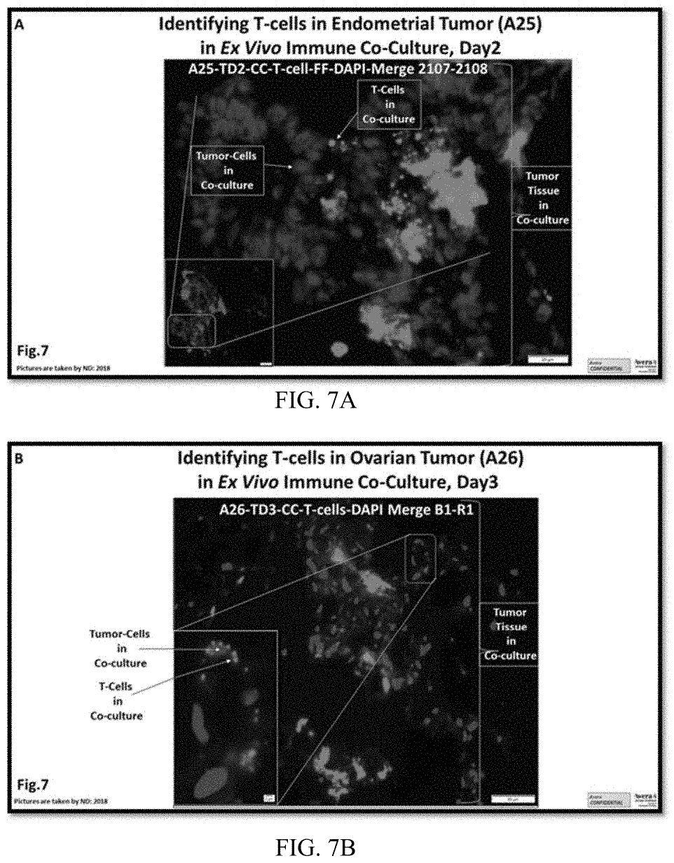

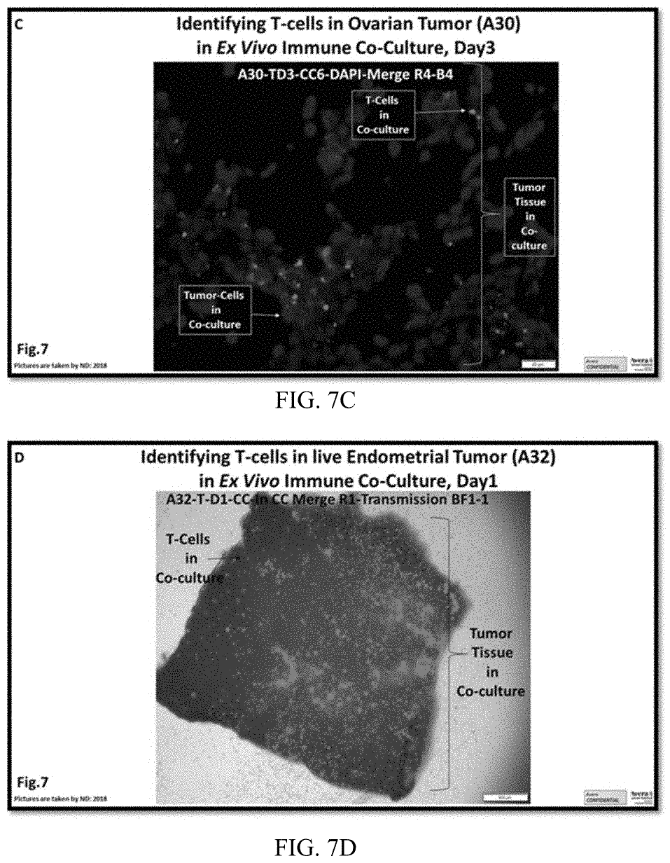

### Example 7—Purification and Identification of T-Cells in Ex-Vivo Immune Co-Culture of Tumor Tissues as Presented in the FIG. 7: Patients #A25, A26, A30, & A32

Method: T-cells were purified from 20 ml (2×10 ml K2EDTA tubes from BD Biosciences) of peripheral blood collected (under fasting condition) from patients on the day of surgery. The purification was carried out using CD3 coated magnetic beads procured from a commercial source (Miltenyi Biotec). The purification was verified by flow cytometry. The purified T-cells were used for co-culture following vital-stain, Dil. Tumor tissue from the patient was co-cultured with the isolated and purified CD3+ T-cells from the patient's peripheral blood after staining it with Dil. Dil-stained (confirmed by flow-cytometry) T-cells (from peripheral blood) were identified in co-culture as well as in fresh frozen sections of harvested co-cultured tissue at different days of co-culture after stained with DAPI.

Media: The 5M media was used and contains DMEM/F-12, 1× Glutamax, 20% FBS, 1% P/S, 1×ITS, 3% BSA, 1% HEPES, and Matrigel growth factor enriched.

Drug(s): No drugs were used during initial standardization.

Results: FIGS. 7A through 7D show the results from this example.

T-cells in the ex-vivo co-culture of tumor tissue are presented in FIGS. 7A, 7B, 7C, and 7D. Identified Dil-stained T-cells in Endometrial Tumor (A25) in Ex-vivo Immune Co-Culture, Day 2 are shown in FIG. 7A. DAPI is used as a counter-stain on the fresh frozen section of the co-cultured tumor tissue (FIG. 7A). Identified Dil-stained T-cells in Ovarian Tumor (A26) in Ex-vivo Immune Co-Culture, Day 3 are shown in FIG. 7B. DAPI is used as a counter-stain on the fresh frozen section of the co-cultured tumor tissue (FIG. 7B). Identified Dil-stained T-cells in Ovarian Tumor (A30) in Ex-vivo Immune Co-Culture, Day 3 are shown in FIG. 7C. DAPI is used as a counter-stain on the fresh frozen section of the co-cultured tumor tissue (FIG. 7C). Identifying Dil-stained T-cells in Endometrial Tumor (A32) in live Ex-vivo Immune Co-Culture, Day 1 are shown in FIG. 7D. The picture was taken from the tissue in co-culture shown in FIG. 7D.

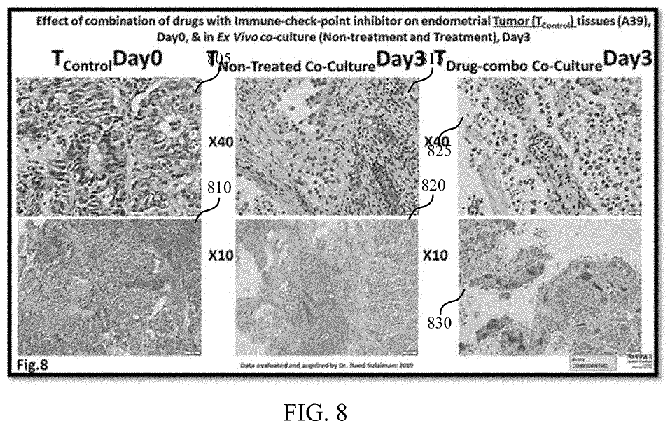

### Example 8—Drug Induced Apoptosis in Ex-Vivo Immune Co-Culture of Endometrial Tumor Tissues as Presented in the FIG. 8: Patients #A39

Method: Endometrial tumor tissue (pT1bN0) was immune co-cultured in the presence of purified and pre-stained T-cells from the same patient's peripheral blood. Histology was performed on non-treated co-cultured tumor tissues and was compared to the drug-treated tissues in the co-cultures at day 0 and day 3 of the co-culture.

Media: The 5M media was used and contains DMEM/F-12, 1× Glutamax, 20% FBS, 1% P/S, 1×ITS, 3% BSA, 1% HEPES, and Matrigel growth factor enriched.

Drug(s): Five drug combinations (paclitaxel; paclitaxel and BKM120; paclitaxel and lenvatenib; paclitaxel and TAK228; paclitaxel and trametinib) were used for testing the drug-induced apoptosis in the tissues in ex-vivo culture. For immune ex-vivo co-culture FDA approved pembrolizumab was used.

Results: FIG. 8 shows the results from this example.

Testing the effect of immuno-therapy drug-combos on Tumor tissue in ex-vivo co-culture for Day 3 is illustrated in FIG. 8. A drug containing immune-check-point inhibitor was tested on both endometrial Tumor (T_{Drug-Combo}) in ex-vivo co-cultures for Day 1, Day 2, and Day 3 as compared to non-treated (NT) tissue on corresponding days. The drug-induced apoptosis has been morphologically identified in tumor tissues, Tumor (T_{Drug-Combo}) as nuclear-debris in contrast to Tumor Day 0 (TD0), as well as non-treated Tumor control (T_{Control}). Representative pictures of the only Day3 with or without drugs (immune-check-point inhibitor) in tumor tissue samples are provided. Tumor (T_{Control}) tissues are found viable in ex-vivo co-cultures for Day 3 as compared to non-cultured tissues (TD0) as shown in photomicrographs 805 and 810. Tumor (T_{Control}) (NT) tissues are shown in photomicrographs 815 and 820 and drug treatment (T_{Drug-Combo}) are shown in photomicrographs 825 and 830. Drug-induced apoptotic changes were observed in photomicrographs 825 and 830 in ex-vivo co-cultures for Day 3 as compared to non-treated (NT) tumor tissues as shown in photomicrographs 815 and 820.

An active proliferation of tumor cells was evaluated morphologically by a certified onco-pathologist who was blind to the drug treatment by the presence of “mitotic figure” for example, as evident from H&E stain as well as in certain cases for example, from IHC stain for the presence of Ki67 in tumor cells. An apoptosis of tumor cells was evaluated morphologically by a certified onco-pathologist who is blind to the drug treatment by the presence of “apoptosis specific nuclear morphology” for example, as evident from H&E stain as well as in certain cases for example, from IHC stain for the presence of cleaved-caspase3 in tumor cells. Tumor-adjacent normal tissue and non-tumor cells and/or tumor stroma of the tumor tissue were used to compare the drug effect.

It should be noted that the methods, systems and devices discussed above are intended merely to be examples. It must be stressed that various embodiments may omit, substitute, or add various procedures or components as appropriate. For instance, it should be appreciated that, in alternative embodiments, the methods may be performed in an order different from that described, and that various steps may be added, omitted or combined. Also, features described with respect to certain embodiments may be combined in various other embodiments. Different aspects and elements of the embodiments may be combined in a similar manner. Also, it should be emphasized that technology evolves and, thus, many of the elements are exemplary in nature and should not be interpreted to limit the scope of the invention.

Specific details are given in the description to provide a thorough understanding of the embodiments. However, it will be understood by one of ordinary skill in the art that the embodiments may be practiced without these specific details. For example, well-known processes and techniques have been shown without unnecessary detail in order to avoid obscuring the embodiments.

Also, it is noted that the embodiments may be described as a process which is depicted as a flow diagram or block diagram. Although each may describe the operations as a sequential process, many of the operations can be performed together, separately, in parallel or concurrently. In addition, the order of the operations may be rearranged. A process may have additional steps not included in the figure.

As used herein, including in the claims, “or” as used in a list of items (e.g., a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

Having described several embodiments, it will be recognized by those of skill in the art that various modifications, alternative constructions, and equivalents may be used without departing from the spirit of the invention. For example, the above elements may merely be a component of a larger system, wherein other rules may take precedence over or otherwise modify the application of the invention. Also, a number of steps may be undertaken before, during, or after the above elements are considered. Accordingly, the above description should not be taken as limiting the scope of the invention.

1. A method for patient-specific evaluation of cancer-therapeutic agents, comprising:
obtaining tumor- and tumor-adjacent normal tissues from a patient;
exposing the tumor- and tumor-adjacent normal tissues to at least one cancer-therapeutic agent;
co-culturing the tumor- and tumor-adjacent normal tissues with the at least one cancer-therapeutic agent;
evaluating the tumor- and tumor-adjacent normal tissues for an effect of the at least one cancer-therapeutic agent; and
selecting at least one cancer-therapeutic agent based on at least one effect.

2. The method of claim 1, wherein the tumor- and tumor-adjacent normal tissues are obtained by surgical resection and/or biopsy from the patient.

3. The method of claim 1, wherein the tumor- and tumor-adjacent normal tissues are resected and/or biopsied from the margin of a tumor of the patient.

4. The method of claim 1, further comprising:
obtaining CD3+ T-cells and/or CD14−/CD15+/CD16+ neutrophils from a blood sample obtained from the patient;
exposing the tumor- and tumor-adjacent normal tissues and the CD3+ T-cells and/or CD14−/CD15+/CD16+ neutrophils to at least one cancer-therapeutic agent; and
co-culturing the tumor- and tumor-adjacent normal tissues and the CD3+ T-cells and/or CD14−/CD15+/CD16+ neutrophils with the at least one cancer-therapeutic agent.

5. (canceled)

6. The method of claim 5, wherein CD3+ T-cells and/or CD14−/CD15+/CD16+ neutrophils are obtained from a peripheral blood sample from the patient.

7. The method of claim 6, wherein CD3+ T-cells and/or CD14−/CD15+/CD16+ neutrophils are obtained from blood from the patient by isolation using magnetic beads.

8. The method of claim 4, wherein the at least one cancer-therapeutic agent is a chemotherapeutic agent, a pathway-targeted drug, an immune-modulatory drug, or any combination thereof.

9. The method of claim 8, wherein the at least one cancer-therapeutic agent may include one or more of carboplatin, paclitaxel, pembrolizumab, copanlisib, everolimus, BYL719, rucaparib, olaparib, talazoparib, niraparib, trametinib, selumetinib, cobimetinib, lenvatinib, pazopinib, venetoclax, cetuximab, earlotinib, afatinib, osimertinib, ceritinib, alectinib, carfilzomib, and TAK228.

10. The method of claim 4, wherein the tumor- and tumor-adjacent normal tissues are co-cultured for a period of at least about 6 hours with the at least one cancer-therapeutic agent, and CD3+ T-cells and/or CD14−/CD15+/CD16+ neutrophils are stained.

11. The method of claim 1, wherein the tumor- and tumor-adjacent normal tissues are co-cultured for a period of at least about 12 hours with the at least one cancer-therapeutic agent.

12. The method of claim 1, wherein the tumor- and tumor-adjacent normal tissues are co-cultured for a period of at least 24 hours with the at least one cancer-therapeutic agent.

13. The method of claim 1, wherein the tumor- and tumor-adjacent normal tissues are co-cultured for a period of at least 48 hours with the at least one cancer-therapeutic agent.

14. The method of claim 1, wherein the tumor- and tumor-adjacent normal tissues are co-cultured for a period of at least 72 hours with the at least one cancer-therapeutic agent.

15. The method of claim 1, wherein evaluating the tumor- and tumor-adjacent normal tissues for an effect of the at least one cancer-therapeutic agent on the tumor- and tumor-adjacent normal tissues includes evaluating at least one of inhibition of proliferation, induction of apoptosis, inhibition of angiogenesis, evaluation of vascular mimicry, status of pathway activation, and immune-status of the tumor- and tumor-adjacent normal tissues.

16. The method of claim 1, wherein selecting the at least one cancer-therapeutic agent based on the at least one effect includes selecting at least one cancer therapeutic agent which induced apoptosis and/or inhibited proliferation of the tumor tissues.

17-30. (canceled)

31. The method of claim 1, wherein the step of co-culturing the tumor- and tumor-adjacent normal tissues with the at least one cancer-therapeutic agent comprises incubating the cultures and co-cultures in hypoxic, reoxygenation, or hyperoxic environments.

32. The method of claim 4, wherein the blood sample obtained from the patient is further tested for the presence and/or absence of circulating tumor cells.

33. The method of claim 4, wherein the blood sample obtained from the patient is further tested for the presence and/or absence of cancer associated macrophage like cells.

34. The method of claim 1, further comprising the steps of:
preparing patient-derived cells from the tumor- and tumor-adjacent normal tissues obtained from the patient; and
conducting a genetic analysis of the tumor tissues to characterize the tumor type.

35. The method of claim 34, further comprising the step of exposing the patient-derived cells to chemotherapeutic agents selected following the steps of claim 1 and/or to therapeutic agents associated with the treatment of the tumor type.
